(19) United States
(12) Patent Application Publication
(10) Pub. No.: US 2019/0287644 A1
(43) Pub. Date: Sep. 19, 2019

(54) Correlation Method To Identify Relevant Genes For Personalized Treatment Of Complex Disease

(71) Applicant: Northeastern University, Boston, MA (US)

(72) Inventors: Alain Karma, Boston, MA (US); Marc Santolini, Allston, MA (US)

(21) Appl. No.: 16/277,216

(22) Filed: Feb. 15, 2019

Related U.S. Application Data
(60) Provisional application No. 62/631,628, filed on Feb. 16, 2018.
(60) Provisional application No. 62/631,454, filed on Feb. 15, 2018.

(51) Int. Cl. G16B 20/00; G16B 20/20

(52) U.S. Cl. CPC G16B 20/00; G16B 20/20

(57) ABSTRACT

Disclosed are novel, personalized bioinformatics correlation methods and systems to reveal a multitude of genes associated with complex diseases. An example method of identifying relevant genes correlated to phenotypic responses to treatment of complex diseases includes acquiring data, for a plurality of individuals, representing biomolecular expressions and phenotypic responses to a treatment. The data for each individual includes a biomolecular expression and phenotypic response pair. The method further includes calculating, across the plurality of individuals, correlation coefficients between the biomolecular expressions and the phenotypic responses to obtain observed correlation coefficients. The method further includes randomizing pairings between the biomolecular expressions and the phenotypic responses and recalculating the coefficients to obtain randomized correlation coefficients. The observed correlation coefficients are compared with the randomized correlation coefficients to create enrichment data, and, based on the enrichment data and the biomolecular expressions, a plurality of relevant genes correlated to the phenotypic responses are identified.

### RELATED APPLICATIONS

This application claims the benefit of U.S. Provisional Application No. 62/631,628, filed on Feb. 16, 2018, and U.S. Provisional Application No. 62/631,454, filed on Feb. 15, 2018. The entire teachings of the above applications are incorporated herein by reference.

### GOVERNMENT SUPPORT

This invention was made with government support under Grant Nos. 5R01HL114437-02 and 5R01HL05242 awarded by the National Institutes of Health. The government has certain rights in the invention.

### BACKGROUND

A traditional approach to investigate the genetic basis of complex diseases is to identify genes with a global change in expression between diseased and healthy individuals. However, population heterogeneity may undermine the effort to uncover genes with significant but individual contribution to the spectrum of disease phenotypes within a population.

### SUMMARY

Presented herein are novel, personalized bioinformatics correlation methods and systems to reveal a multitude of genes associated with complex diseases. One example embodiment is a method of identifying relevant genes correlated to phenotypic responses to treatment of complex diseases (e.g., administration of a drug to an individual). The example method includes acquiring data, for a plurality of individuals, representing biomolecular expressions and phenotypic responses to a treatment. The data for each individual includes a biomolecular expression and phenotypic response pair. The method further includes calculating, across the plurality of individuals, correlation coefficients between the biomolecular expressions and the phenotypic responses to obtain observed correlation coefficients. The method further includes randomizing pairings between the biomolecular expressions and the phenotypic responses and recalculating the coefficients to obtain randomized correlation coefficients. The observed correlation coefficients are compared with the randomized correlation coefficients to create enrichment data, and, based on the enrichment data and the biomolecular expressions, a plurality of relevant genes correlated to the phenotypic responses are identified. The identified genes can be used to develop personalized treatment of a complex disease.

Another example embodiment is a system for identifying relevant genes correlated to phenotypic responses to treatment of complex diseases. The example system includes an interface, memory, and a processor in communication with the interface and memory. The interface is configured to acquire data, for a plurality of individuals, representing biomolecular expressions and phenotypic responses to a treatment. The data for each individual includes a biomolecular expression and phenotypic response pair, and is stored in the memory. The processor is configured to calculate, across the plurality of individuals, correlation coefficients between the biomolecular expressions and the phenotypic responses to obtain observed correlation coefficients. The processor is further configured to randomize pairings between the biomolecular expressions and the phenotypic responses and recalculate the coefficients to obtain randomized correlation coefficients. The processor is further configured to compare the observed correlation coefficients with the randomized correlation coefficients to create enrichment data, and identify, based on the enrichment data and the biomolecular expressions, a plurality of relevant genes correlated to the phenotypic responses.

Another example embodiment is a machine-readable storage medium having stored thereon a computer program for identifying relevant genes correlated to phenotypic responses to treatment of complex diseases. The computer program comprises a routine of set instructions for causing the machine to acquire data, for a plurality of individuals, representing biomolecular expressions and phenotypic responses to a treatment. The data for each individual includes a biomolecular expression and phenotypic response pair. The computer program further comprises a routine of set instructions for causing the machine to (i) calculate, across the plurality of individuals, correlation coefficients between the biomolecular expressions and the phenotypic responses to obtain observed correlation coefficients, (ii) randomize pairings between the biomolecular expressions and the phenotypic responses and recalculate the coefficients to obtain randomized correlation coefficients, (iii) compare the observed correlation coefficients with the randomized correlation coefficients to create enrichment data, and (iv) identify, based on the enrichment data and the biomolecular expressions, a plurality of relevant genes correlated to the phenotypic responses.

Acquiring data can include acquiring biomolecular expressions from the individuals and evaluating phenotypic responses of the individuals before and after treatment. Biomolecular expression for an individual can include biomolecular expression data before treatment of the individual, or can include a fold change of biomolecular expression data before and after treatment of the individual, in which case the fold change can be a ratio of the biomolecular expression after and before treatment of the individual. A phenotypic response for an individual can be computed from measurements of the phenotype before and after treatment of the individual. The phenotypic responses can be, for example, binary or of a continuous spectrum of phenotypic responses.

Calculating the correlation coefficients can include, in the case in which biomolecular expression for an individual includes biomolecular expression data before treatment of the individual, calculating correlation coefficients between the biomolecular expression data before treatment of the individuals and the phenotypic responses. Alternatively, in the in the case in which biomolecular expression for an individual includes a fold change of biomolecular expression data before and after treatment of the individual, calculating the correlation coefficients can include calculating correlation coefficients between the fold changes of biomolecular expression data and the phenotypic responses.

In some embodiments, the biomolecular expressions can be filtered to exclude biomolecular expressions that are of lower confidence. Alternatively, acquiring data can include acquiring an electronic representation of the biomolecular expressions, such as via a network connection to a non-transitory computer readable medium, such as a web-based server.

Comparing the observed correlation coefficients with the randomized correlation coefficients to create enrichment data can include dividing a proportion of the observed correlations by a proportion of the randomized correlations.

In some embodiments, the enrichment data can include an enrichment curve as a function of the observed correlation coefficients ordered by decreasing values. In such embodiments, identifying the plurality of genes relevant to the phenotypic responses can include identifying the plurality of genes based on observed correlation coefficients from the start of the enrichment curve to the peak of the enrichment curve.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1L illustrate two types of responses to stressor-induced heart failure. FIG. 1A illustrates histograms of pre-ISO (blue) and post-ISO (red) heart masses of Hybrid Mouse Diversity Panel (HMDP) strains. FIG. 1B illustrates typical Differentially Expressed Genes (DEGs), which show clear population-average fold-change allowing distinguishing of two populations of strains. FIG. 1C illustrates an example of strong DEG, namely Serpina3n. FIG. 1D is a histogram of heart mass fold-changes (FC) computed for each strain from the HMDP. FIG. 1E illustrates that an expression FC at the individual level can lead to cases were the population-average FC is null while the individual FCs are not. FIG. 1F illustrates Kcnip2, an example of a gene with no population-wide average FC. FIG. 1G illustrates how at the individual level, Kcnip2 shows strong variations, as seen in the histogram of individual FCs at the strain level (log 2 of post over pre-ISO expression ratio). Some strains have a 4-fold decrease of expression (−2 in log 2), while others have a 4-fold increase (+2). FIG. 1H illustrates the strain-specific heart mass FC by decreasing strength. Red bars indicate increase in value and blue bars indicate decrease in value. FIG. 1I illustrates Serpina3n log FC by the same strain ordering of FIG. 1H. Its population-wide FC is high (3.9), with most strains showing a strong positive FC (red bars). FIG. 1J illustrates the correlation of Serpina3n FC with the heart mass FC being not significant (r=−0.09, p=0.43). FIG. 1K illustrates Kcnip2 having a weak population-wide FC (FC=0.85). Some strains show an increased expression (red bars) while others show a decreased expression (blue bars). The red arrow indicates the 129X1/SvJ strain in which Kcnip2 has previously been shown to be down-regulated during cardiac hypertrophy. FIG. 1L illustrates Kcnip2 FC being significantly correlated to heart mass FC (r=0.4, p=1.5e-4), with increased expression corresponding to high hypertrophy and decreased expression corresponding to low hypertrophy.

FIGS. 2A-2F illustrate identification of genes associated with severity of cardiac hypertrophy. FIG. 2A is a histogram of the absolute values of the correlations between FCs of genes expression and hypertrophy for all genes (blue, observed, red, randomized phenotype). Individual FCs are more correlated to hypertrophy than expected. The inset plot corresponds to the best observed correlation. FIG. 2B illustrates assessment of previous enrichment by computing the ratio of the area under the observed and randomized curve as a function of correlation cutoffs. Cutoffs are matched to the genes correlations ranked in decreasing order. The enrichment peaks at N=36 genes, which defines the set of “FC” genes. FIG. 2C is a boxplot comparing the values of the absolute correlation with hypertrophy for the 2,538 SAM genes resulting from a population-wide DEG study and for the 36 identified individual FC genes. FC genes have significantly higher correlation. FIG. 2D is a heatmap showing the 36 genes (columns) log fold-changes across strains (rows). The left column shows the degree of hypertrophy (yellow=low, dark blue=high). Hierarchical clustering shows a natural grouping of the strains by the severity of hypertrophy. FIG. 2E illustrates enrichment of 36 best FC genes in human disease genes from GWAS studies. The 15 most enriched sets are shown. Red arrows indicate cardiac diseases. The enrichment of the 36 best SAM genes is shown for comparison, with low enrichment in the found sets. FIG. 2F illustrates enrichment of 36 SAM genes. These genes show enrichment in “Fibrosis,” a feature of structural remodeling during cardiac hypertrophy.

FIGS. 3A-3E illustrate FC genes that are co-regulated and significantly connected to the cardiac hypertrophy signaling network (CHSN). FIG. 3A illustrates co-expression networks of the 36 best FC and SAM genes in healthy and post-ISO hypertrophic strains. Edges are drawn between two genes if the square Pearson correlation is greater than 0.1 (r²>0.1). The two modules segregate naturally using a force layout algorithm, showing that the modules have high clustering but only few links between themselves. Interestingly, Nppb (purple arrow) segregates with SAM genes, especially in ISO condition. FIG. 3B illustrates edge density of the FC module, the SAM module, and the FC to SAM. Edges are computed and compared to the density expected for random sets of nodes of the same size. The corresponding Z scores are significantly high (Z>2) for both modules, indicating high co-expression. However, there are significantly fewer links than expected between the two modules (Z<−2), indicating that they are disjoint in the co-expression network. FIG. 3C illustrates the 6 most enriched TF motifs in the −/+20 kb regions around the 36 FC genes TSSs predicted using iRegulon. Interestingly, Snai3 (blue arrow) is a SAM gene and Hes1 (red arrow) a FC gene, suggesting a crosstalk between the two modules at the gene regulatory level. FIG. 3D illustrates a proportion of neighbors in the interactome that belong to the Cardiac Hypertrophy Signaling Network (CHSN) for different gene sets: the FC set (red arrow), the 36 best SAM genes (blue arrow) and 1,000 realizations of random nodes in the interactome with the same size as the FC set (gray histogram). Z scores are computed relative to the gray distribution. The FC set is significantly connected to the CHSN, while the SAM genes are not significantly different than a random set. FIG. 3E illustrates a network visualization of the CHSN, along with neighbors from the 36 best FC genes (red nodes).

FIGS. 4A-4C illustrate validation of Hes1 as a new cardiac hypertrophy regulator. FIG. 4A illustrates Hes1 mRNA expression following 48 h after siRNA transfection in a control, isoproterenol or phenylephrine medium. Three siRNAs were used, a scrambled, control one and two Hes1 specific siRNAs. Both Hes1 siRNAs show systematic downregulation of Hes1 mRNA in all conditions. FIG. 4B illustrates an effect of Hes1 knockdown on the known hypertrophic makers Nppa and Nppb. In both case, Hes1 knockdown leads to a significant change in biomarkers activation in isoproterenol and phenylephrine conditions (*p<0.05, ***p<1e-3, Student t-test). FIG. 4C illustrates an effect of Hes1 knockdown on neonatal rat ventricular myocytes size relative to control medium cell cross-sectional area. Both siRNAs lead to a drastic 80-90% decrease in hypertrophy in both isoproterenol and phenylephrine media.

FIG. 5 is a flow diagram illustrating a method of identifying relevant genes correlated to phenotypic responses to treatment of complex diseases, according to an example embodiment.

FIG. 6 is a schematic view of a computer network environment in which the example embodiments presented herein may be implemented.

FIG. 7 is a block diagram illustrating an example computer node of the network of FIG. 6.

### DETAILED DESCRIPTION

A description of example embodiments follows.

Many common diseases are influenced by a combination of multiple genes and environmental factors. These diseases are referred to as complex diseases. Presented herein are novel, personalized (also referred to as “precision”) bioinformatics correlation methods and systems to reveal a multitude of genes associated with complex diseases. The results, validated experimentally by in vitro knockdown, demonstrate that individualized approaches are useful to unmask all genes involved in complex diseases, opening new avenues for the development of personalized therapies.

The disclosed methods and systems use personalized as opposed to population-level gene expression and phenotypic data. For example, one method can compute the correlation between the personalized fold change of gene expression and the personalized fold change of any suitable continuously varying scalar quantity characterizing the change of phenotype (e.g., change of heart mass in cardiac hypertrophy) in response to a perturbation (e.g., pharmacological treatment inducing hypertrophy). Responses can be objective (quantitative) or subjective (qualitative). The method can identify relevant genes as genes whose expression fold-change have a statistically significant correlation (e.g., determined by randomization of gene expression and phenotypic data) to the fold-change of the scalar quantity characterizing the change of phenotype. An example advantage of the disclosed methods and systems is that they do not assume homogeneity across individuals as traditional population-level methods of differential gene expression. The disclosed methods and systems can be embodied in bioinformatics software, for example, to identify relevant genes in complex diseases and develop personalized pharmacological treatments and gene therapies.

The following described a particular example of identifying relevant genes correlated to phenotypic responses to treatment of complex diseases.

### INTRODUCTION

Contrary to “Mendelian” diseases where causality can be traced back to strong effects of a single gene, common diseases result from modest effects of many interacting genes. Understanding which genes are involved and how they affect diseases is a major challenge for designing appropriate therapies.

Heart failure (HF) is a well-studied example of a genetically complex disease involving multiple processes that eventually lead to a common phenotype of abnormal ventricular function and cardiac hypertrophy. Numerous studies have attempted to pinpoint differentially expressed genes (DEGs) to find biomarkers for the prognosis of the disease and the design of appropriate drugs, as well as explore underlying affected signaling pathways. Such studies typically compare the average gene expression between samples in healthy and diseased states, such as non-failing versus failing hearts in murine, canine, or human samples. Genes are ranked by the strength of their differential expression, and top-ranking genes are further investigated for pathway enrichment and biomarker potential. However, because of the different genetic backgrounds of the surveyed individuals, as well as different severities of HF, those studies show very limited overlap of DEGs. While separate studies typically identify tens to hundreds of DEGs, not a single DEG is common to all studies. Moreover, it is unclear whether the healthy state is itself a well-defined unique state. In particular, several studies have shown that, due to compensatory mechanisms involved in homeostasis, different combinations of ion channel conductances in neurons and cardiac cells can lead to a normal electrophysiological phenotype, e.g., a similar bursting pattern of motor neurons or a similar cardiac action potential and calcium transient. This has led to the concept that genetically distinct individuals represent different “Good Enough Solutions” corresponding to distinct gene expression patterns underlying a healthy phenotype. Different combinations of gene expression in a healthy state resulting from genetic variations would be expected to yield different DEGs in a diseased state. Thus, small numbers of DEGs that are only shared by a subset of individuals, and would be missed by a standard population-wide DEG analysis, could in principle have a causal role. Identifying these genes remains a central challenge in personalized medicine.

In order to explore the variability of individual trajectories leading to hypertrophy and HF, the following leverages the Hybrid Mouse Diversity Panel (HMDP), a model system consisting of >100 genetically diverse strains of mice (see Methods, below). Gene expression and phenotypic data are acquired before and three weeks after implantation of a pump delivering isoproterenol (ISO). This pathological stressor induces a global response characterized mainly by cardiac hypertrophy along with more marginal changes in chamber dilation and contractile function at the population level. As a result, primary focus was on the identification of genes relevant for cardiac hypertrophy. Expression data is collected at the whole heart level and the Total Heart Weight is used to quantify the degree of cardiac hypertrophy. Importantly, the severity of the hypertrophic response is highly variable among strains, ranging from almost no hypertrophy to up to an 80% increase of heart mass. The study is directed at understanding why certain individuals are more susceptible to or protected against cardiac hypertrophy due to their genetic backgrounds. Because mice from the same strains are isogenic and renewable, the HMDP offers the possibility to analyze differential gene expression and phenotype evolution in a unique setting where subjects in the control population can be matched to a subject with the same genetic background in the diseased populations. In that setup, one can correlate the stressor-related gene expression change with phenotype change (in the example case, heart mass increase) while controlling for genetic background, thereby disentangling intra-strain (stressor-induced) and inter-strain (genetics-induced) variations. In the specific case of HF which is the focus in this example, such data could not be obtained in human studies where heart tissue biopsies are extracted from either healthy donor hearts or explanted hearts of late stage HF patients in a genetically diverse population. One would indeed require a population of identical twins in which one twin for each pair of twins is a heart donor and the other twin is a late stage HF patient. As such, gene expression data obtained from those biopsies can only be used to perform a population-level differential gene expression analysis. In contrast, relevant genes are identified by correlating strain-specific temporal changes of gene expression, i.e., differential expression between a post-ISO mouse and another pre-ISO mouse from the same strain, with the corresponding strain-specific changes of phenotype, i.e., ratio of heart mass between the post- and pre-ISO mice of the same strain.

Concretely (see Methods, below), the Pearson coefficient of correlation c₁ are calculated between the strain-specific fold-change of expression of gene j among N different strains

$${F}_{j} = ( \frac{{E}_{1}^{′} ( j )}{{E}_{1} ( j )} , \frac{{E}_{2}^{′} ( j )}{{E}_{2} ( j )} , … , \frac{{E}_{N}^{′} ( j )}{{E}_{N} ( j )} ) ,$$

where Eᵢ(j) and E′ᵢ(j) are the expression levels of gene j for two isogenic mice of the i^{th} strain before and after ISO treatment, respectively, and the strain-specific fold-change of heart mass among different strains

$${F}_{m} = ( \frac{{m}_{1}^{′}}{{m}_{1}} , \frac{{m}_{2}^{′}}{{m}_{2}} , … , \frac{{m}_{N}^{′}}{{m}_{N}} ) ,$$

where mᵢ and m′ᵢ are the total heart mass of isogenic mice of the i^{th} strain before and after ISO treatment, respectively. This method of differential gene expression analysis identifies a set of DEGs, referred to hereafter as “fold-change” (FC) genes, for which the absolute value of cⱼ is above a threshold of statistical significance determined by randomization of the data as detailed further in Methods, below. The ability to study a large number (N˜100) of strains using the HMDP is essential to have enough statistical power to establish such a correlation, a power that has been lacking from previous studies limited to small numbers of strains. Moreover, the correlation coefficients cⱼ cannot be calculated in the setting of traditional clinical studies since the fold change of gene expression or heart mass of subjects with different genetic background is meaningless. Conversely, it is possible to analyze the HMDP data set using the same type of population-level differential gene expression analysis used in clinical studies, such as SAM (Significance Analysis of Microarrays). Applied to the HMDP data set, a method like SAM identifies a gene j as differentially expressed if the expression data in the control population (E₁ (j), E₂ (J), . . . , E_{N}(j)) and the treated population (E′₁(j), E′₂(j), . . . , E′_{N}(j)) originate from different distributions. Comparing those two distributions is fundamentally different than calculating the correlation coefficients cⱼ between Fⱼ and Fₘ.

Based on the computation of the cⱼ correlation coefficients, a small set of 36 FC genes are found and compared to a larger set of genes identified with SAM (referred to hereafter as SAM genes). Interestingly, the sets of FC and SAM genes have negligible overlap. The FC genes are not identified as significantly changed at the population level because they typically have opposite fold changes in low and high hypertrophy strains that cancel each other when averaged over all strains in the population-wide case. It is shown that the FC genes are strongly enriched in cardiac disease genes from previous Genome-Wide Association Studies (GWAS), while SAM genes are in contrary enriched in fibrosis genes. It is also shown that those two sets form two distinct communities in the co-expression network among healthy as well as ISO-injected strains, and potential Transcription Factors (TFs) to explain the observed co-regulation of FC genes are identified. Moreover, it was found that the proteins encoded by the FC genes, but not the SAM genes, interact predominantly with proteins belonging to a cardiac hypertrophic signaling network (CHSN) that has been shown to provide a predictive model of hypertrophy in relation to multiple stressors including ISO. Interestingly, it was found that one of the FC genes, namely Hes1, is also a predicted TF and an important interactor with the CHSN. Using a knockdown approach, it was found that it plays a major role in cardiac hypertrophy, allowing us to validate the personalized, multi-omics approach.

Results

1. Two Types of Responses to Stressor-Induced Cardiac Hypertrophy and Heart Failure

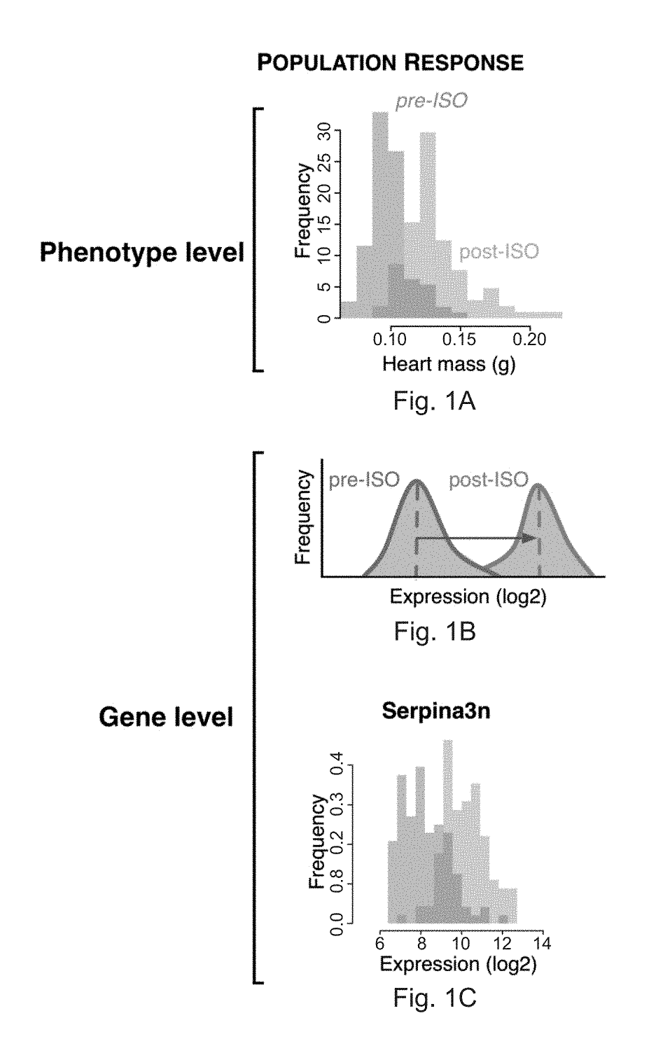

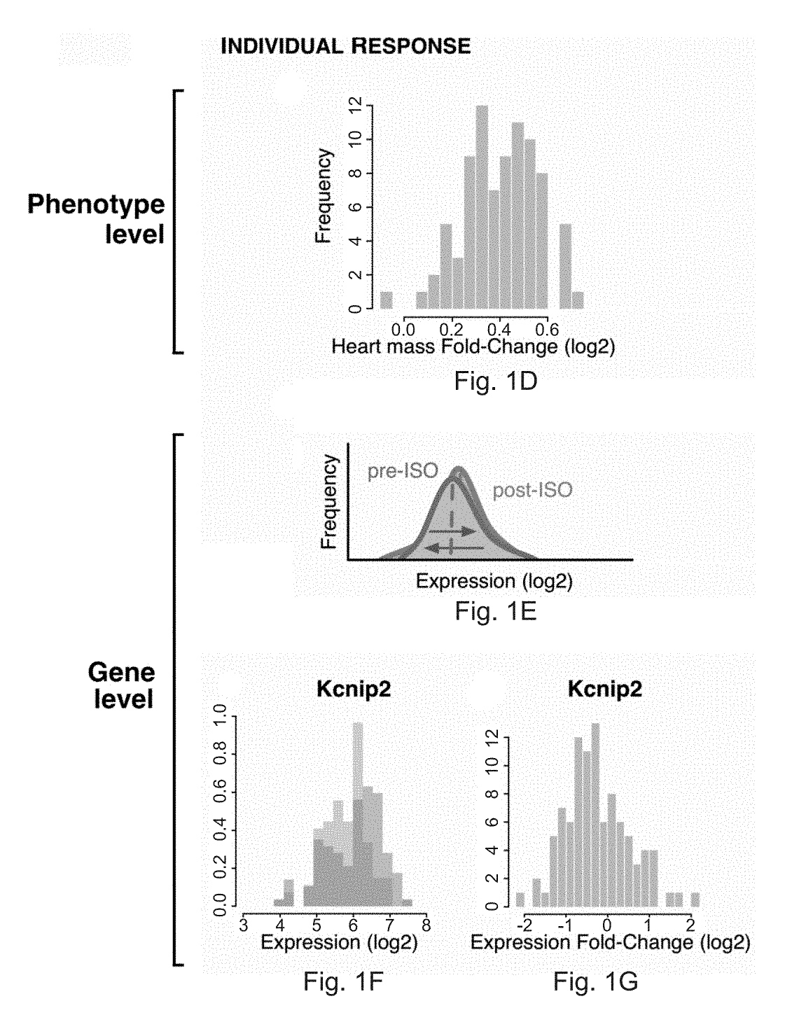

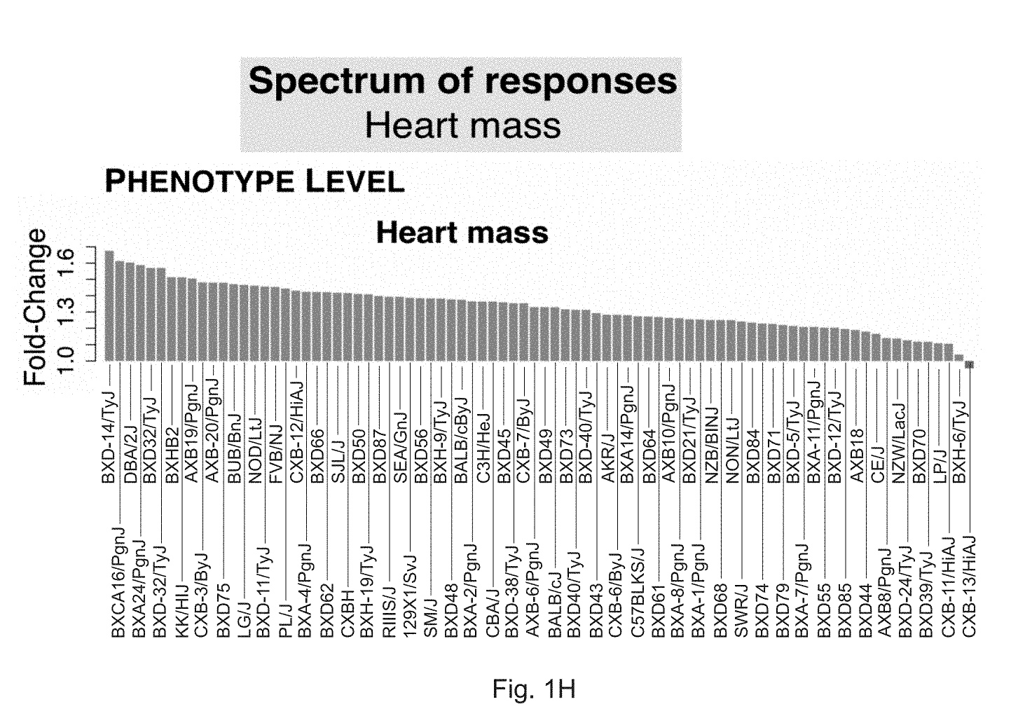

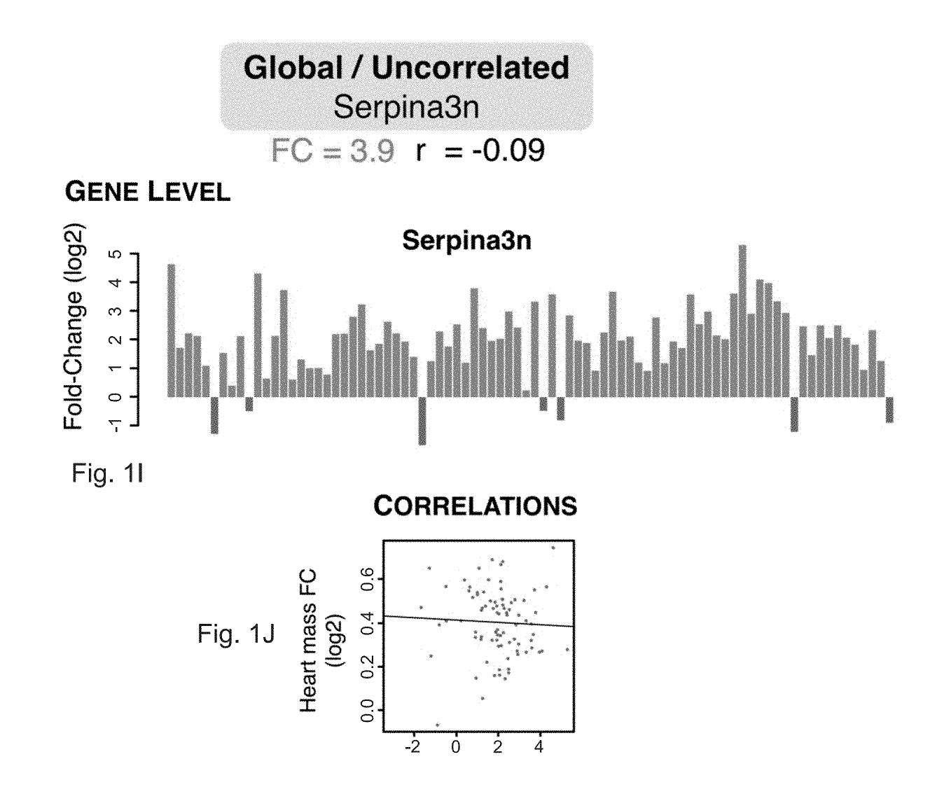

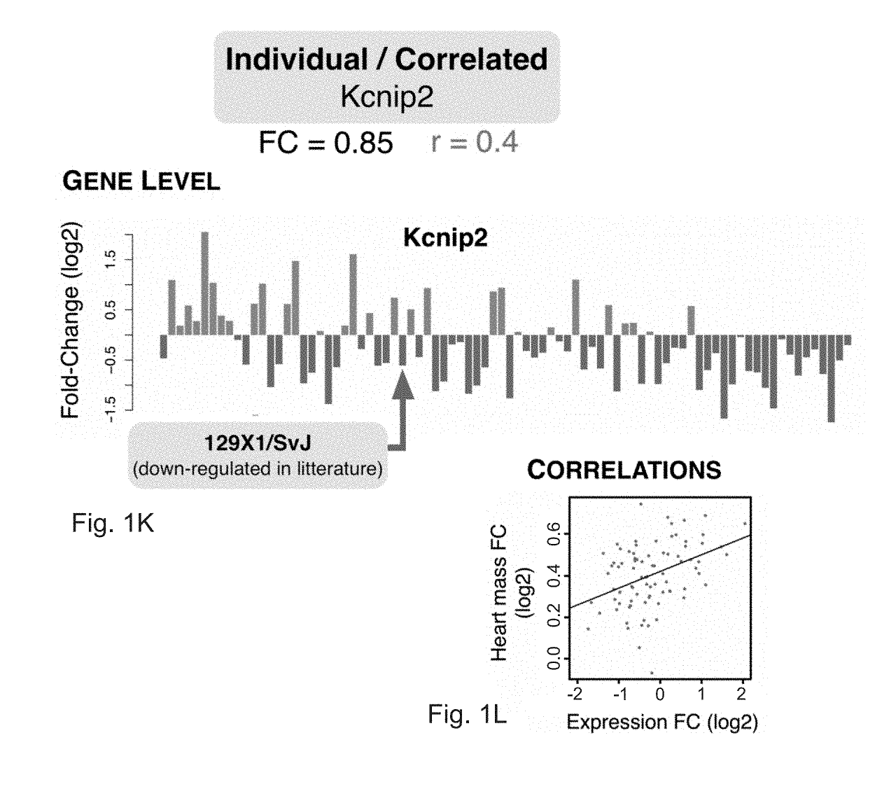

One example shows two distinct ways to describe the response to ISO in the HMDP (see FIG. 1A-1G and Methods). First, it is noted that ISO induces a global response across all strains, resulting in cardiac hypertrophy. This is seen in FIG. 1A, where the distribution of heart mass among the post-ISO strains can clearly be distinguished from the pre-ISO distribution (p<2.2e-16 under Student t-test). At the gene level, such a response is typically analyzed by looking for DEGs at the population level, i.e., genes for which the change in average expression with the stressor is significantly greater than the variability with and without the stressor (see FIG. 1B). Typical tools include t-test, SAM, or LIMMA. Genes found with these methods have a differential expression profile at the population level and are therefore potential biomarkers of the trait of interest (see microarray data for Serpina3n, ranked #13 by SAM, in FIG. 1C). However, despite the global response in the level of gene expression to ISO, the degree of hypertrophy among individual strains is highly variable, from almost none to an 80% increase of heart weight (see FIG. 1D). This calls for an evaluation of the strength of differential gene expression at the individual level. In particular, a whole new class of genes becomes available for analysis. Indeed, even if a gene does not show population-wide average differential expression, it can show extensive variation at the individual, strain-specific level (see FIG. 1E). This is the case for the gene Kcnip2 encoding the protein KChIP2, which interacts with pore forming subunits (Kv4.2 and Kv4.3) of the transient outward current Iₜₒ expressed in heart, and which has been implicated in cardiac hypertrophy. Though not showing population-wide differential expression (see FIG. 1F), its individual fold-change of expression can vary drastically from 2-fold decrease to a 2-fold increase depending on the considered strain (see FIG. 1G). Interestingly, when comparing the individual variations of those two types of genes with the degree of hypertrophy (see FIGS. 1H-1K), it can be seen that global DEGs are not necessarily good descriptors of the individual changes of phenotype (see FIG. 1J), unlike the second type of genes missed by a traditional population-wide method (see FIG. 1L). In particular, in the case of Kcnip2, a significant positive correlation with the severity of hypertrophy (r=0.4, p=1.5e-4) was observed. This is particularly interesting since Kcnip2 has previously been shown to be down-regulated during cardiac hypertrophy in the strain 129X1/SvJ. While this finding was confirmed, it was also observed that it is unusual in a broader context, and that Kcnip2 is most of the time up-regulated in strains with marked hypertrophy.

In the following, these observations are generalized to identify a larger set of genes that, like Kcnip2, have an individual FC correlated to the severity of hypertrophy, and this set was compared to the complete set of DEGs identified by the population-level SAM method.

2. Identification of Genes Associated with the Severity of Hypertrophy

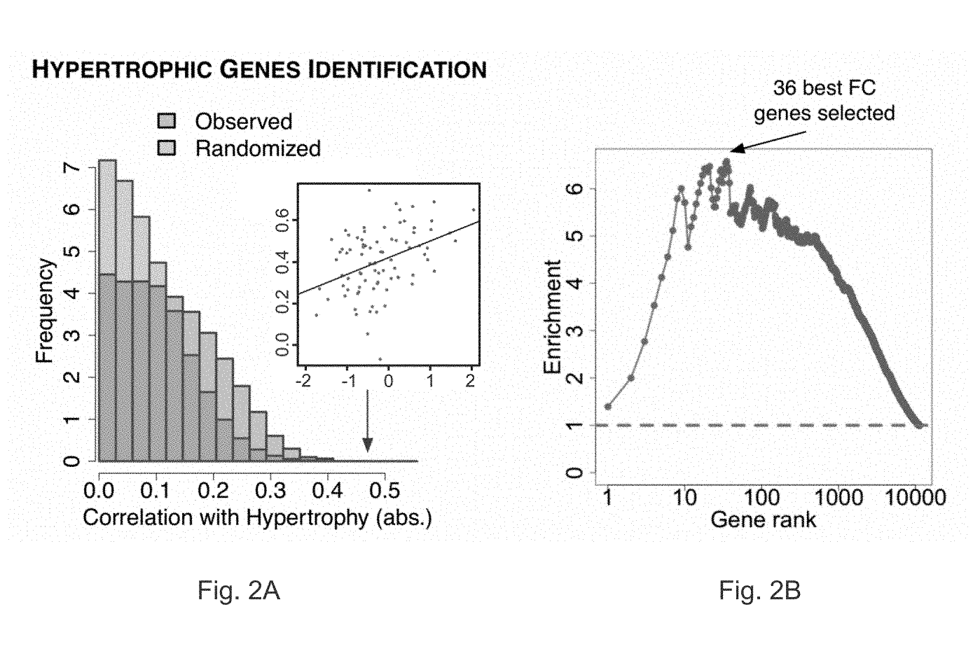

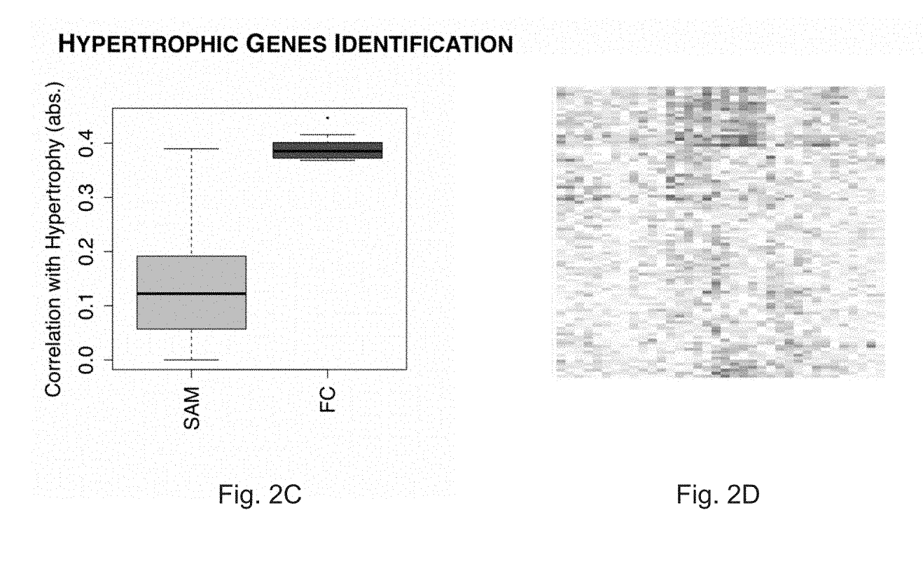

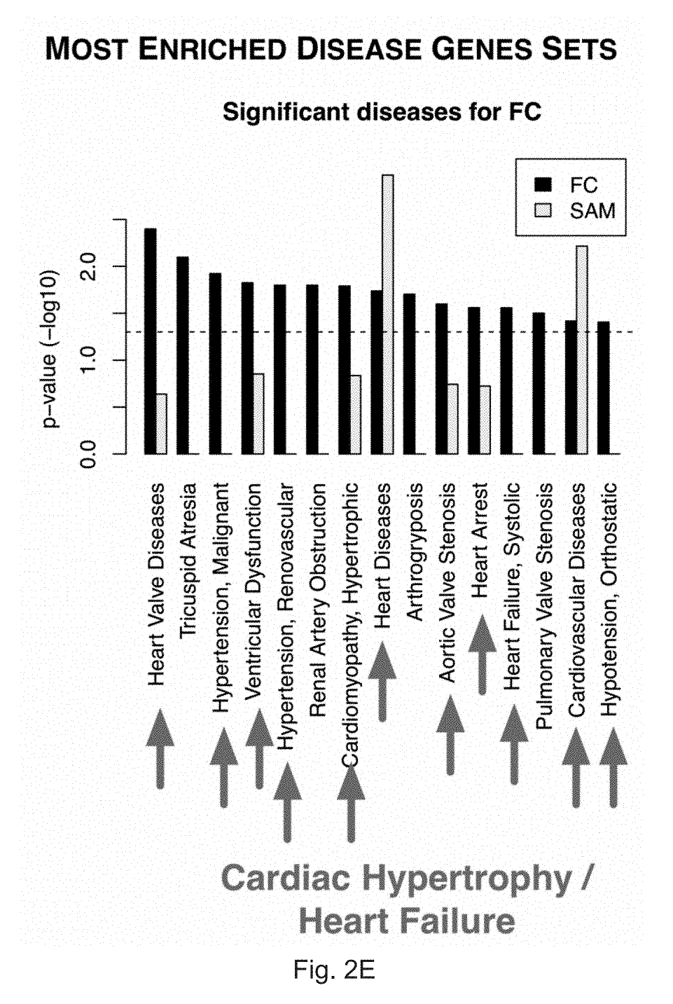

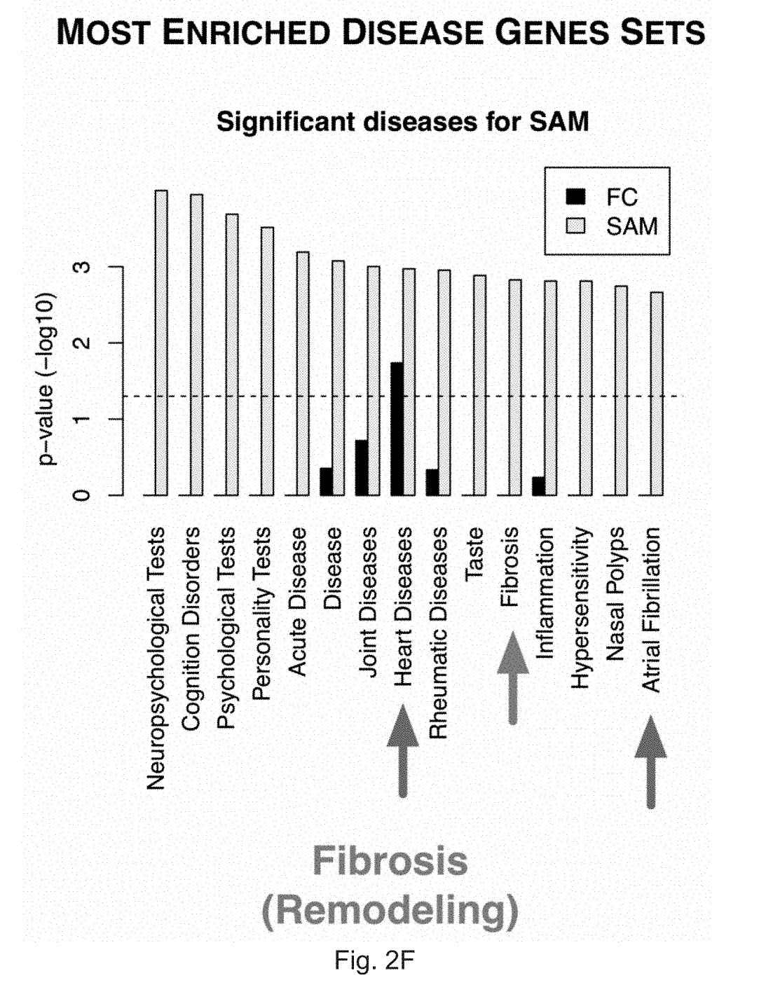

Described herein is an example method to determine which genes show individual, strain-specific expression FCs significantly correlated to the individual hypertrophic response measured by the individual fold-change of heart mass. Since the methodology is based on correlations, those genes are selected that belong to the giant component of the gene co-expression network above a certain correlation cutoff (see Methods, below). The advantages of such a filter compared to one based on absolute expression levels is that it yields a clear, well-defined cutoff while also rejecting genes having high expression but artefactual correlations (e.g., hitting a microarray saturation level). Obtained is a filtered set of 11,279 high-confidence genes. Then, the absolute Pearson correlation is computed for all genes between the gene expression fold-change and the individual hypertrophic response (see FIG. 2A, blue histogram). To control for false positives, the expected correlations when randomizing the phenotype by shuffling strain labels are computed (see Methods and FIG. 2A, red histogram). One can see significant enrichment in genes with high correlation to the trait. To quantify this enrichment, the proportion of observed (‘blue’) correlations divided by the proportion of correlations in the randomized cases (‘red’) above various correlation cutoffs can be computed. FIG. 2B shows this enrichment as a function of the gene rank, ordered by decreasing absolute value of the correlation with hypertrophy. The enrichment shows a peak at 36 genes, followed by a plateau until about 500 genes, and a subsequent decrease. These 36 genes can be defined as candidates to describe the hypertrophic spectrum. These genes are listed in Table 1, along with references supporting the involvement of several of them in cardiac hypertrophy and HF. In the following, this set of genes is referred to as the “FC” set.

TABLE 1
List of genes predicted with the individual Fold-Change analysis

| Rank | Gene | Correlation | Known role from literature |
|---|---|---|---|
| 1 | Rff1 | −0.446656 | Hypertension |
| 2 | Wdr1 | 0.415692 | Cardiac Hypertrophy |
| 3 | Nppb | 0.408886 | Heart failure marker |
| 4 | Atp6v0a1 | 0.407205 | Hypertension |
| 5 | Ankrd1 | 0.406246 | Dilated cardiomyopathy |
| 6 | Eif4a1 | 0.404824 | |
| 7 | Dtr (HB-EGF) | 0.403043 | Heart failure |
| 8 | Kcnip2 | 0.402246 | Downregulated in hypertrophy |
| 9 | Pcdhgc4 | −0.402053 | |
| 10 | Hes1 | 0.400076 | Heart outflow tract development |
| 11 | 4930504E06Rik | 0.396189 | |
| 12 | Akap9 | −0.389713 | LQT syndrome |
| 13 | 2310022B05Rik | 0.3897 | |
| 14 | Bclaf1 | −0.388563 | |
| 15 | Ttc13 | −0.387981 | |
| 16 | Nipsnap3b | 0.387325 | |
| 17 | Gss | 0.386407 | Glutathione Synthetase, linked |
| | | | to cardiac abnormalities |
| 18 | Klhl23 | −0.385625 | |
| 19 | Tspan17 | 0.384865 | |
| 20 | Tnni2 | −0.383516 | |
| 21 | Cab39l | −0.381902 | |
| 22 | Ptrf (Cavin-1) | 0.381134 | Dilated cardiomyopathy |
| 23 | Dedd | 0.378059 | |
| 24 | 9430041O17Rik | 0.375683 | |
| 25 | Fgf16 | 0.373829 | Heart disease |
| 26 | Ehd2 | 0.372787 | Regulate cardiac membrane |
| | | | protein targeting |
| 27 | Ppp1r9a | −0.372641 | Subunit of the same complex |
| | | | than PPP1R3A, involved in |
| | | | HF in human patients |
| 28 | Kremen | 0.372366 | Interacts with Wnt signaling |
| 29 | Scara5 | −0.372294 | |
| 30 | Zfp523 | −0.372223 | |
| 31 | Nfatc1 | 0.371409 | Cardiac hypertrophy |
| 32 | Corin | −0.369546 | Cardiac protease that regulates |
| | | | blood pressure by activating |
| | | | natriuretic peptides, involved |
| | | | in cardiac hypertrophy |
| 33 | Prnpip1 | 0.369466 | |
| 34 | Lrrc1 | 0.369161 | |
| 35 | AW549877 | −0.368865 | |
| 36 | Mkrn3 | −0.368269 | |

As a comparison, the population-wide DEGs using Significance Analysis of Microarray or SAM was computed. This exhibited 2,538 DEGs at a False Discovery Rate of 1e-3 (see Methods, below). Interestingly, no significant overlap (p=0.68, hypergeometric test) was found between these SAM genes and the FC set, with six genes common to both sets (Tspan17, Ppp1r9a, Bclaf1, AW549877, Gss, 2310022B05Rik and 9430041O17Rikm). In general, correlations between the individual fold-changes of the SAM genes and the degree of hypertrophy are found to be quite low (see FIG. 2C). This shows that population-wide analyses do not naturally yield personalized FC genes, calling for a specific method to uncover them.

The 36 FC genes are shown in FIG. 2D. As expected from the absence of overlap with SAM genes, the FC genes have both negative (blue) and positive (red) fold-change across the different strains, meaning that they have negligible average fold-change at the population level. A question that arises is whether the variability observed in the individual fold-changes of gene expression across strains is a consequence of genetic variability, or merely reflects environmental or experimental spurious effects. To investigate this question, the fact that gene expression has been replicated in 9 strains post-ISO can be leveraged. Since mice from the same strain have a similar genetic background, they should therefore show very comparable individual fold-changes. The replicability was assessed by computing the Spearman rank correlation of the 36 FC genes fold-change profiles between mice from replicated strains. A large mean correlation of 0.76 was found, compared to 0.14 for pairs of strains taken at random among the non-replicated pool with a statistically very significant p-value (p=1.6e-7, Wilcoxon test). This result shows that individual fold-changes are tightly controlled at the genetic level and that the ranking of the genes by FC is preserved for approximately ⅔ of the cases. Replicability was also assessed by making a scatter plot of the log 2 expression fold-change computed with the original and replicated ISO treated hearts compared to the same control heart for the 36 FC genes and 9 strains. A correlation analysis of this scatter plot yields a correlation of 0.57 and very low p-value (p<2.2e-16), confirming that individual fold-changes are predominantly genetically determined.

The following evaluates further the biological signal carried by these FC genes missed by population-wide methods.

3. Biological Relevance of the Identified FC Genes

Given the importance of the genetic control of those genes, they must be more susceptible to genetic variations. To explore that idea, the enrichment in disease genes coming from previous GWAS can be evaluated. The HuGE database of human genes associated with 2,711 different diseases can be used (see Methods, below). First, the mouse gene names are converted to human as described in Methods. Then, the diseases are ranked according to their enrichment in 36 FC (resp. 36 best SAM) genes using a hypergeometric test assuming as null hypothesis a uniform repartition of the genes across diseases. Results are shown in FIGS. 2E and 2F for the 15 most enriched diseases in each case. It was observed that FC genes are strongly enriched in heart diseases (11 in the 15 most enriched diseases) while SAM genes are only enriched in two cardiac diseases and in fibrosis, a feature characteristic of the structural remodeling taking place during HF. Those findings exhibit two distinct roles of FC and SAM genes in the progression of cardiac hypertrophy. While the cross-talk between cardiac fibroblasts and myocytes during cardiac hypertrophy has been studied previously, here, their relative contributions are disentangled into a shared, population-wide fibroblastic component, and a fine-tuned, individualized component capable of explaining the severity of cardiac hypertrophy. Moreover, the enrichment of FC genes in human GWAS genes also highlights the relevance of the present HMDP data analysis to human cardiac hypertrophy and HF.

4. Co-Expression and Co-Regulation

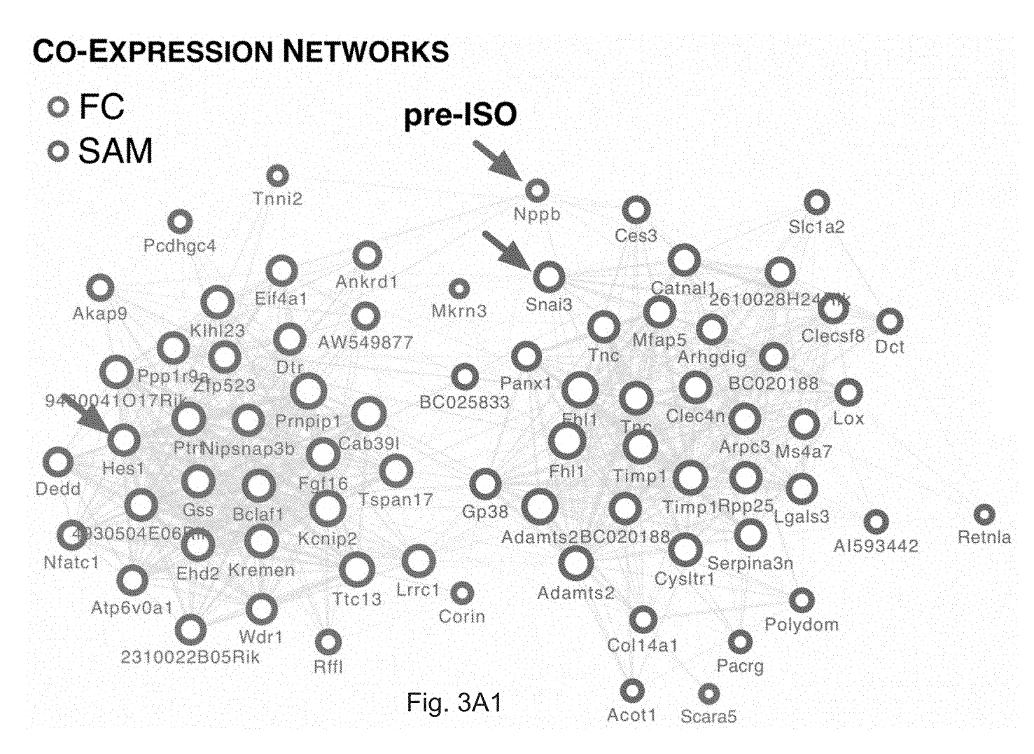

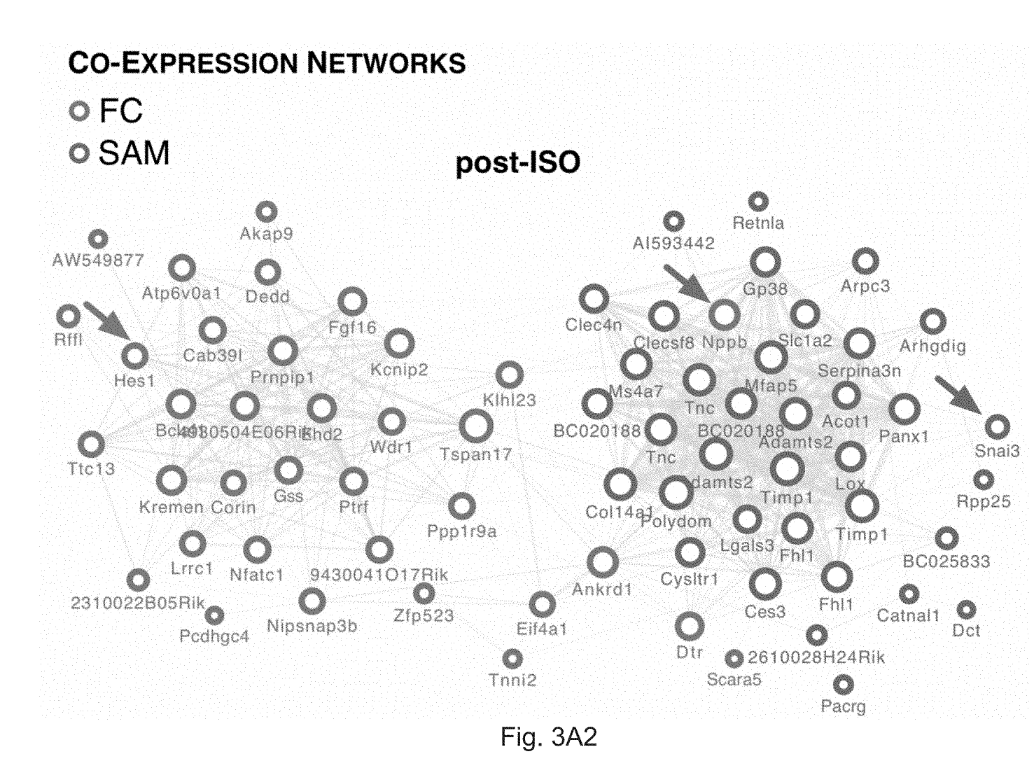

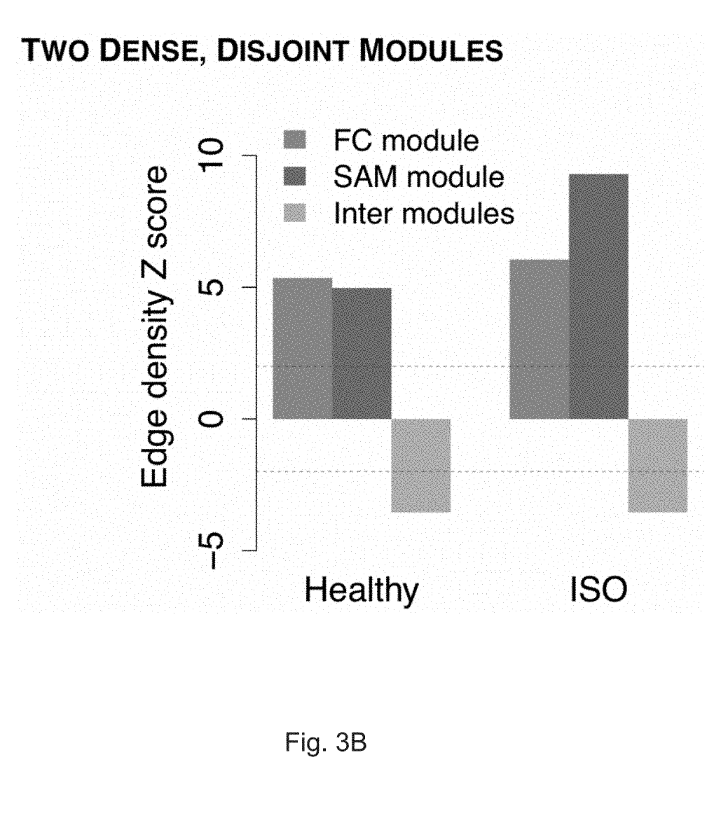

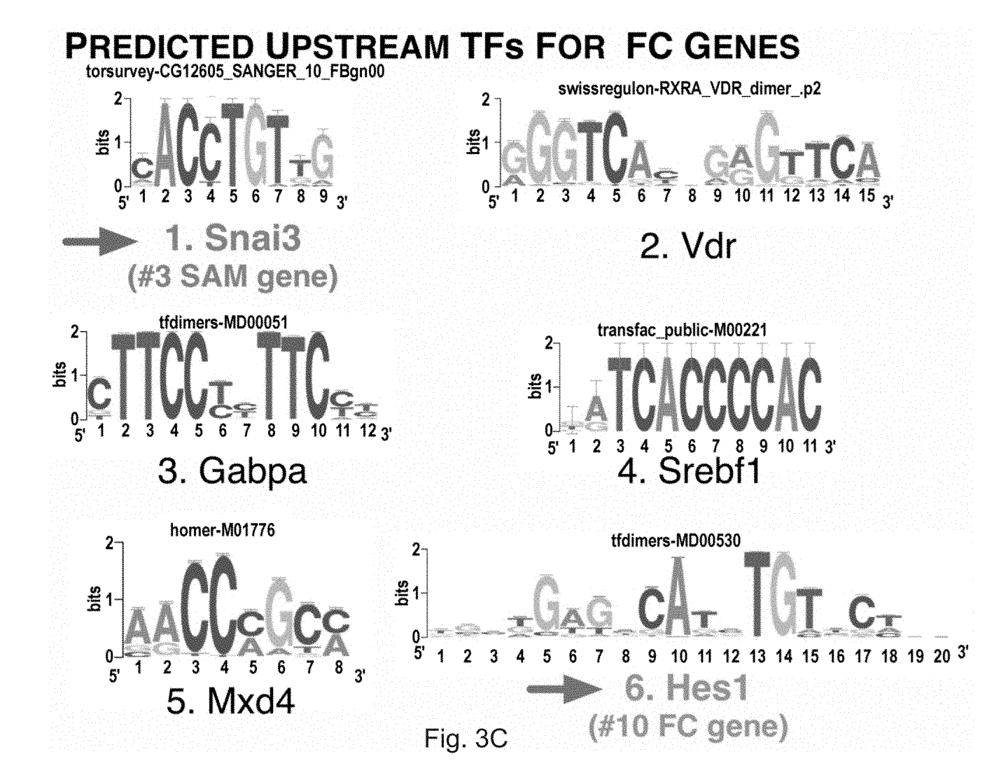

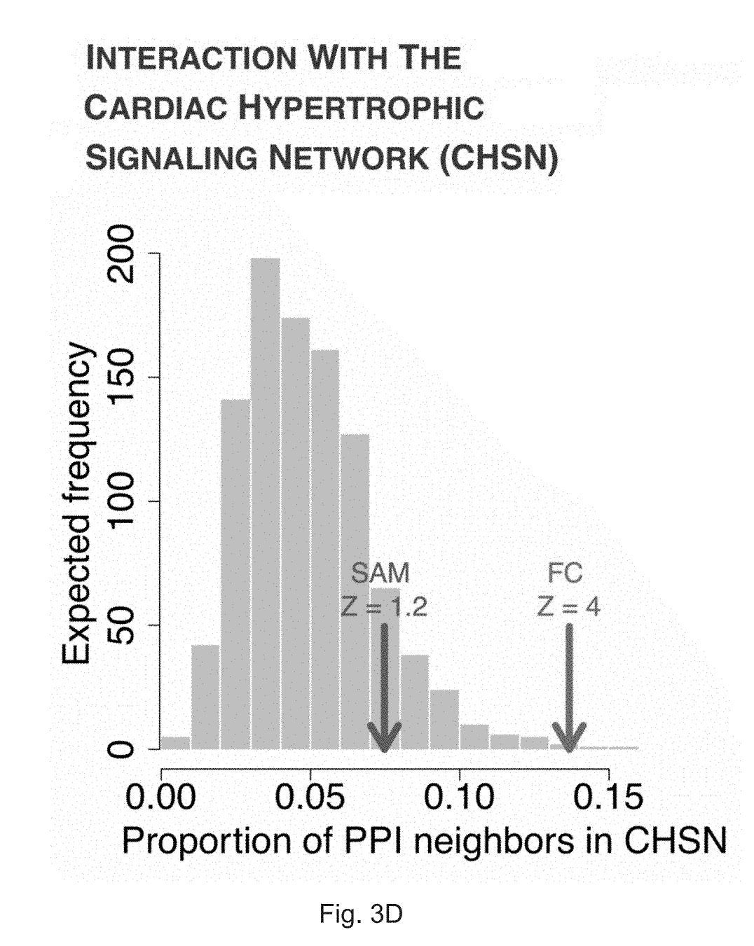

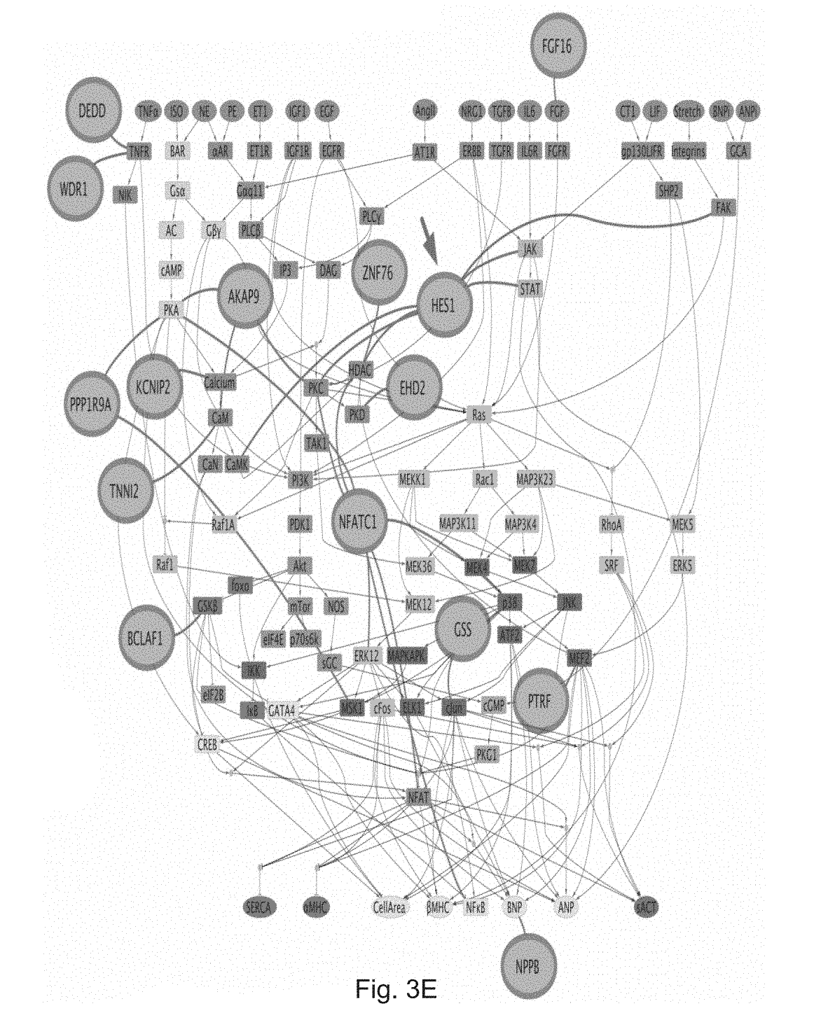

The identified sets of population-level and individual FC genes have until now been considered as collections of independent genes. However, in the cell, genes function together to achieve higher-order physiological functions. Such a collective behavior can be assessed in the framework of co-expression networks, where genes are related by the similarity of their profile of expression across different conditions. In the context of the HMDP, it is investigated herein whether the predicted sets of genes show evidence of co-regulation in healthy and post-ISO hypertrophic strains. To that extent, the squared Pearson correlations (r²) is computed between the 36 best genes of both the FC and SAM sets. Correlation matrices are then cut off at r²>0.1 to keep significant interactions. Show in FIGS. 3A and 3B are the resulting co-expression networks in pre- and post-ISO conditions. It is clear that the two sets of genes form dense modules, and are disconnected from each other, with only few links between the two sets. Interestingly, it is shown that the biomarker and modulator of hypertrophy Nppb acts as a bridge between the two modules in pre-ISO condition (see FIG. 3A, top), and is even found strongly co-expressed with the SAM genes in post-ISO mice (see FIG. 3B). This suggests a role for Nppb in driving a cross-talk between FC genes and SAM genes. Finally, to quantify the relative density of the modules, they were compared to 1,000 sets of a similar number of randomly selected genes. The resulting Z scores are shown in FIG. 3C. Both SAM and FC sets show much stronger co-expression than randomly expected, with the SAM module being even denser under ISO condition. On the contrary, the density of links between the two modules is significantly smaller than expected by chance, indicating that the two sets of genes are disjoint sets in the co-expression network. Overall, these results show that the FC and SAM genes form two tight, disjoint communities in the co-expression network, both in pre-ISO and post-ISO mice.

The finding that the FC genes are strongly co-expressed suggests that they are co-regulated. To explore this possibility, enrichment in common TF binding sites was evaluated in the vicinity of the 36 FC genes. To compute the enrichment, iRegulon was used, a recent algorithm integrating different TF motifs databases and using phylogenic conservation to identify overrepresented binding sites in the −20/+20 kb regions around the Transcription Start Sites of genes of interest (see Methods, below). The identified motifs were then ranked by target enrichment among selected genes, and are associated with a list of putative TFs that can bind them (see FIG. 3C). It was found that the best-ranked motif is associated with repressor TFs Scrt1 and Scrt2, known to modulate the action of basic helix-loop-helix (bHLH) TFs. Interestingly, the corresponding PWM motif is also matched to Snai3 TF, a gene ranked third among SAM genes. The second motif, VDR, is known to be involved in heart failure and cardiac hypertrophy. Finally, the sixth predicted TF is associated with Hes1, which ranks tenth among the FC genes. This indicates that there is a cross-talk between the two modules at the gene regulatory level, with both FC and SAM genes being involved in the regulation of the expression of the FC genes.

5. Exploration of the Neighborhood in the Interactome

While useful to detect gene regulatory changes involved in the disease process, gene expression does not capture post-translational changes and interactions that occur at the protein level. To explore the potential involvement of the predicted sets of genes at the protein level, a previously published human interactome was used combining high-throughput and literature curated protein-protein, metabolic, kinase-substrate, signaling, and to a lesser extent regulatory interactions. After converting to human gene symbols (see Methods, below), the proteins encoded by the 36 best FC and SAM genes have respectively 364 and 346 interacting partners. Then, pathway enrichment was computed for these neighbors (see Methods). The other most highly enriched pathway is linked to NFAT signaling, known to be important in HF. Interestingly, it was found that the second most enriched pathway for FC neighbors is a previously published Cardiac Hypertrophy Signaling Network (CHSN) containing 106 nodes (corresponding to 218 genes) giving a predictive model of hypertrophy in response to multiple stressors including ISO. Indeed, about 14% of FC neighbors are components of this network, compared to a predicted random association of 4% (Z=4, see FIG. 3D). The CHSN is shown in FIG. 3E, along with FC nodes directly interacting with CHSN nodes. In particular, it was found that Hes1 is interacting with several nodes of the CHSN at different levels of the hierarchy, namely FAK, JAK, STAT, CamK, PKC and HDAC.

6. Experimental Validation of Hes1

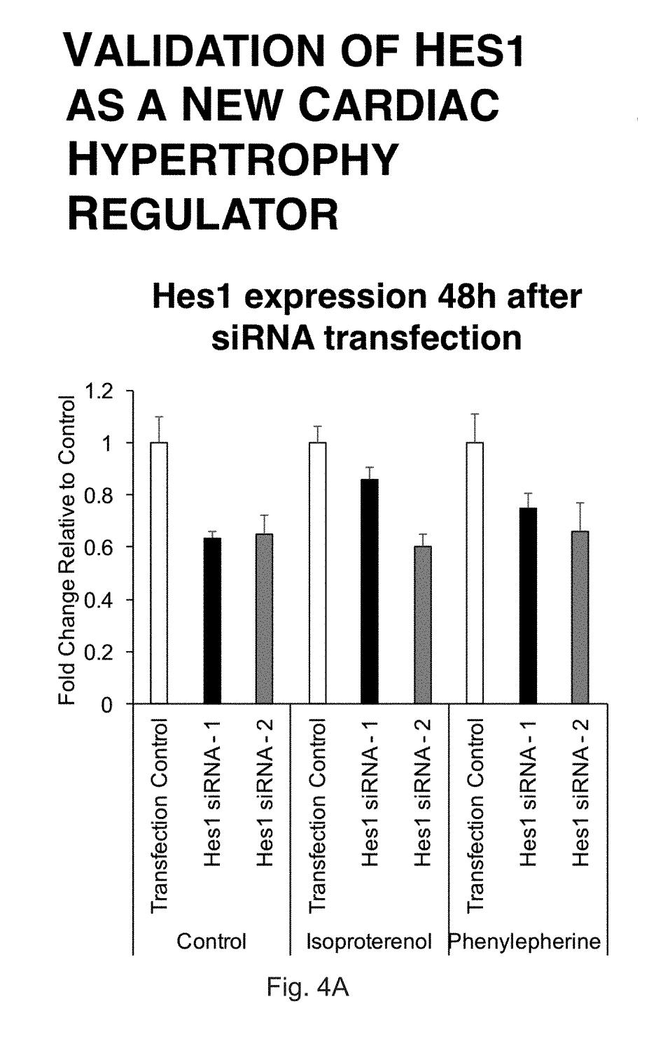

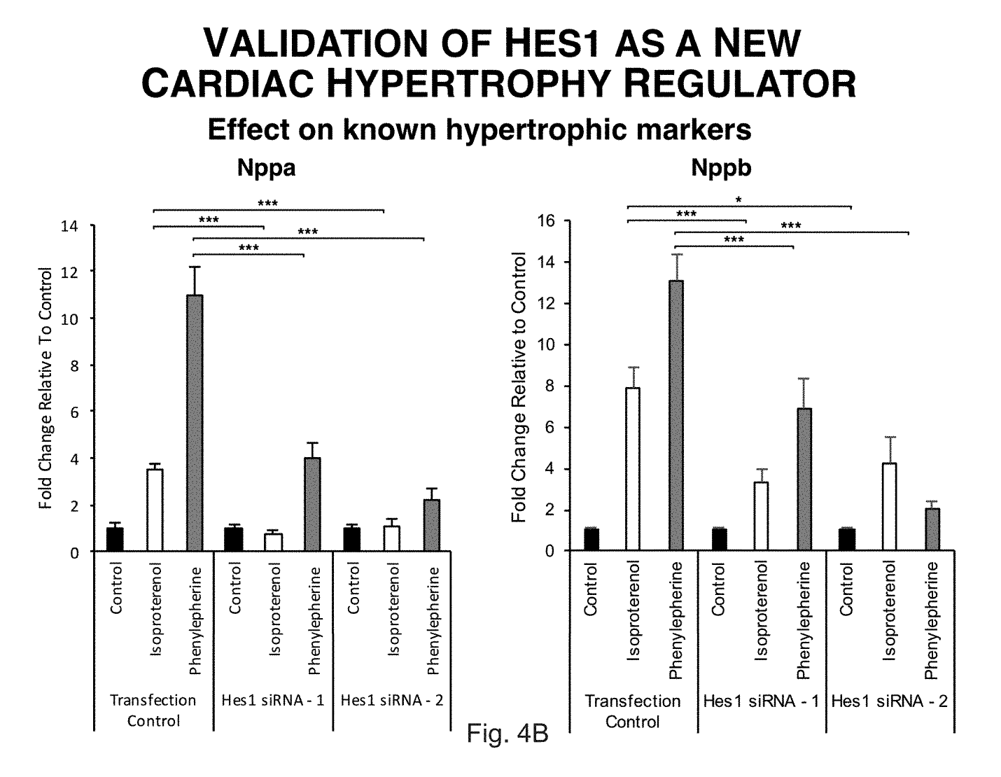

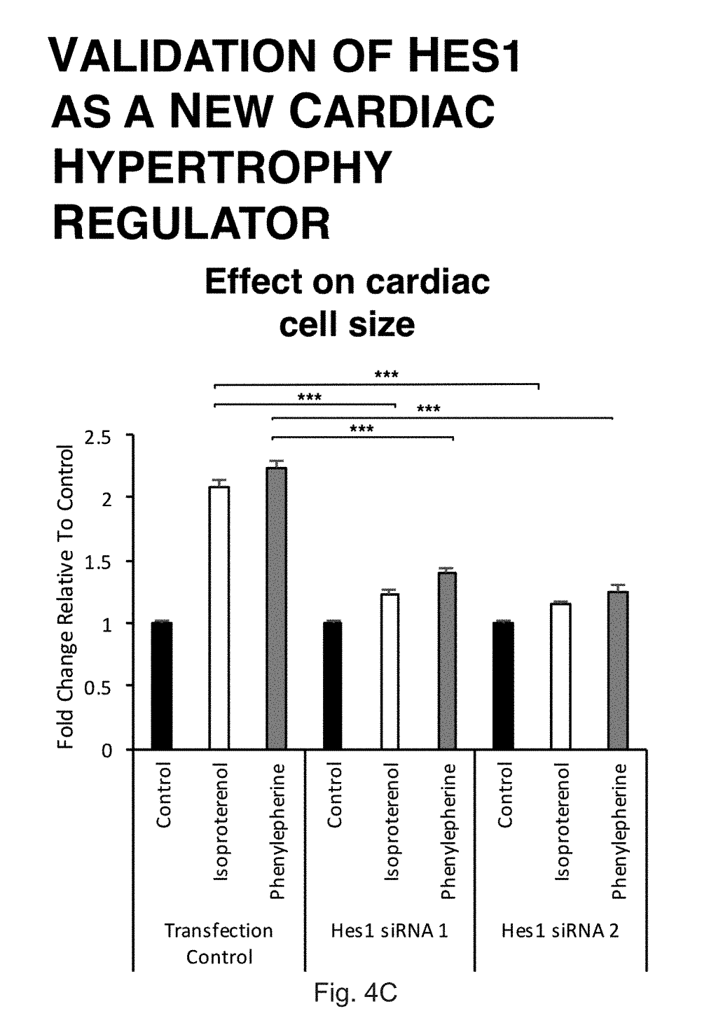

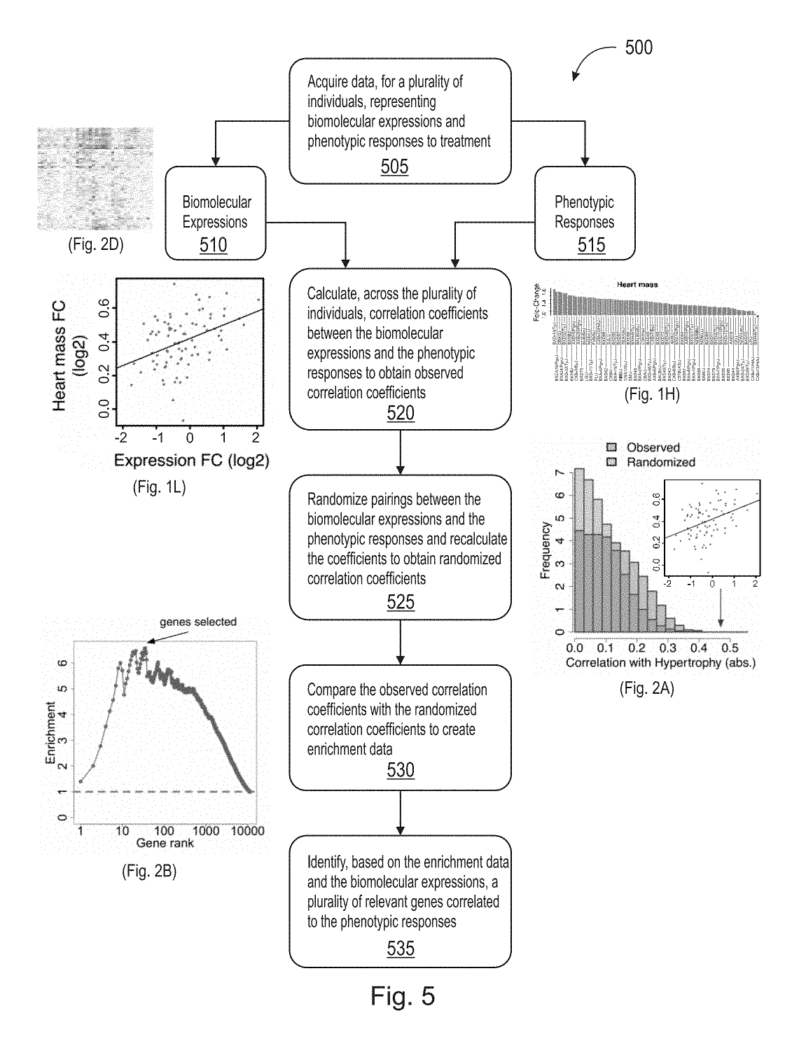

The previous results point toward a role for Hes1 in cardiac hypertrophy and heart failure. Indeed, Hes1 was found to be a FC gene, an upstream regulator of FC genes, and an interactor with several components of the CHSN. To determine the function of Hes1 in the context of cardiac hypertrophy and heart failure, siRNA knockdown in neonatal rat ventricular myocytes (NRVMs) was performed, followed by treatment with beta-adrenergic agonist isoproterenol (ISO) or alpha-adrenergic agonist phenylephrine (PE) containing media. Both agents induce hypertrophy through different molecular pathways, as can be seen in the CHSN (see FIG. 3E). Using siRNA to silence Hes1 expression, a 20% to 40% decrease in Hes1 expression was achieved when compared to transfection control (see FIG. 4A). At the molecular level, treatment with either ISO or PE containing media drastically increases the expression of the HF markers Nppa and Nppb, which rose 3.5- and 7.9-fold, respectively under ISO treatment and 11-fold and 13-fold, under PE treatment in cells transfected with the control siRNA. Strikingly, knockdown of Hes1 expression strongly impaired the induction of these two markers under both treatment conditions. Nppa induction was reduced up to 110% and 88% under ISO and PE treatment while Nppb induction was reduced up to 66% and 91% under ISO and PE treatment, respectively. In addition to these molecular changes, the role of Hes1 in modulating the increase in cardiomyocyte cell cross-sectional area upon treatment with ISO and/or PE was investigated. As expected, following ISO/PE treatment, cells transfected with the control siRNA doubled in cellular cross-sectional area (see FIG. 4C). In comparison, cells transfected with the Hes1 siRNA showed up to 87% and 79% reduction in cell cross-sectional area increase following treatment with ISO and PE, respectively. This effect is consistent with the fact that HMDP strains showing no or mild hypertrophy exhibit strong negative fold-change of Hes1. Taken together, these findings strongly suggest a role for Hes1 as a novel regulator of cardiac hypertrophy in vitro.

7. Discussion

The present study investigated the spectrum of cardiac hypertrophy and HF development in 100+ genetically diverse mice from the HMDP when subjected to chronic ISO infusion. Two types of responses were analyzed. First, the global response at the population level with a large number (1,000+) of genes involved, as detected by the SAM algorithm. Their global fold-change is representative of the global hypertrophy observed across all strains. However, the magnitude of their fold-change at the individual level does not predict the degree of individual hypertrophy. Using a correlation-based method, another group of about 40 genes was found that predicts the degree of hypertrophy. These can be called the “FC” genes in reference of the fact that they were found using their individual, strain-specific fold-change. Surprisingly, these genes have a near zero fold-change at the population level due to the cancelling contributions of up- and down-regulation in different strains, so that they are not detected using classical differential expression tools. While several FC genes have previously been implicated in cardiac hypertrophy and HF (see Table 1, above), their high variability in such a controlled setup has not been explored previously. It is showed herein that these genes are enriched for heart failure gene candidates previously described in the literature, as well as for human cardiac disease genes. On the other hand, the best SAM genes are enriched in fibrosis disease genes. ISO has been shown to induce first myocardial fibrosis concomitantly with myocyte necrosis, followed by myocyte hypertrophy on a longer time scale, and fibrosis is also known to be an early manifestation of hypertrophic cardiomyopathy. The results suggest that population-level SAM genes are predominantly associated with the early fibroblast response. On the other hand, since the change of heart mass is primarily determined by myocyte growth, the results suggest that FC genes are associated with the strain-specific degree of myocyte growth induced by beta-adrenergic stimulation.

The roles of these genes in different biological networks was further investigated. It was found that both FC and SAM genes form distinct co-expressed modules. Interestingly, Nppb (encoding the BNP protein), a widely used biomarker and modulator of HF, belongs to the FC set but is co-expressed with SAM genes in healthy mice, providing a unique bridge between the two sets. This result is consistent with the previous finding that Nppb is an antifibrotic hormone produced by myocytes with an important role as a local regulator of ventricular remodeling in mice. Indeed, Nppb is correlated to the fibrotic SAM genes in healthy mice, consistent with a regulatory homeostatic behavior, but is found among FC genes after beta-adrenergic stimulation, consistent with a response proportionate to myocytes hypertrophy. It is also interesting to note that the SAM module overlaps significantly (p=3.4e-6, hypergeometric test) with a co-expression module previously found in post-ISO mice and shown to be involved in cardiac hypertrophy. Indeed, it shares the genes Timp1, Tnc, Mfap5, Coll4a1 and Adamts2, the latter of which was validated experimentally as a regulator of cardiac hypertrophy.

Several TFs were then predicted to study this co-regulation. Interestingly, among the top TFs predicted as regulators of the FC genes, one of them, Hes1, belongs to the FC genes, and another one, Snai3, belongs to the SAM genes. Both inhibitory (Snai3, Hes1) and activatory (Vdr, Srebf1) TFs were found to have enriched binding sites around FC genes TSSs. This suggests a potential regulatory balance that could explain the up- and down-regulation observed for these genes across strains. Potential post-translational effects at the protein level were evaluated by using an integrated interactome. It was found that FC genes were strongly interacting with a cardiac hypertrophy signaling network (CHSN) previously shown to be predictive of cardiac hypertrophy in response to ISO and other stressors. This may indicate that several of those genes are upstream of a causal chain of events at the post-translational level that control myocyte growth. The FC gene Nppb is present both as an input and an output of the CHSN. This exemplifies an interesting feedback architecture where downstream effects can causally affect upstream regulation. Overall, the FC genes constitute a HF “disease module” formed of co-regulated genes connected to the CHSN at the protein level.

A key finding of the study presented herein is that there is strong strain-to-strain variation in response to a stressor under similar well-controlled environmental conditions. This variation is largely explained by the different genetic backgrounds, as shown by the consistent responses in mice from same strains and the strong enrichment in heart diseases GWAS (see FIG. 2E). For example, Kcnip2 is known to be downregulated concomitantly with a reduction of Iₜₒ magnitude in cardiac hypertrophy. The results are consistent with this finding for the previously studied 129X1/SvJ strain, but show that Kcnip2 is upregulated in many strains with pronounced hypertrophy leading to an overall positive correlation between Kcnip2 expression and heart mass FC. This indicates that there are multiple possible compensatory mechanisms underlying a similar patho-phenotype. Similarly, a strong variation in the fold-change of Nppb was observed. It was previously shown to be over-expressed during cardiac hypertrophy as an anti-fibrotic factor. Using the multiple strains setup presented herein, a positive correlation between Nppb change of expression and the degree of hypertrophy was observed. However, some cases were observed where hypertrophic strains exhibit down-regulation of Nppb, including the widely used C57BL/6J and 129X1/SvJ strains (see FIG. 2D).

Finally, the approach was validated by testing Hes1's role in cardiac hypertrophy. Hes1 was chosen because of its involvement at different levels: found as a FC gene, Hes1 is also a predicted TF regulating the FC genes and a key interactor of the CHSN. Hes1 is part of the Notch signaling pathway which is highly conserved and involved in cell-cell communication between adjacent cells. This pathway is well known to play a crucial role in cardiac development and disease. Notch activity is required in complex organs like the heart that necessitate the coordinated development of multiple parts. Specifically, functional studies have shown that Notch activity is required for cardiovascular development and that Notch signaling causes downstream effects such as cell fate specification, cell proliferation, progenitor cell maintenance, apoptosis, and boundary formation. In previous studies, Hes1 expression was observed to increase following myocardial infarction and other ischemic cardiomyopathies. Increased expression of Hes1 was also shown to inhibit apoptosis of cardiomyocytes and promote instead their viability. However, whether Hes1 acts as a regulator of novel heart failure markers has remained unclear. Here, it is shown show that Hes1 knock-down induces a dramatic reduction of hypertrophy by 80-90% (see FIG. 4C), identifying for the first time Hes1 as a key regulator of cardiac hypertrophy. Importantly, this result is consistent with the HMDP, where strains with no or mild hypertrophy have 20-50% decrease in Hes1 after ISO injection.

Overall, the individual, strain-specific responses to stressor-induced HF were explored and 36 FC genes were identified that are missed by traditional population-wide method of DEG analysis. It is shown that these FC genes provide a completely distinct, albeit complementary, picture of HF than population-wide DEGs. In particular, FC genes are enriched in human cardiac disease genes and hypertrophic pathways. This is important since previous studies that use population-level methods to identify DEGs have concluded that murine models are of limited relevance to human HF. In contrast, the findings show that FC genes, identified by a personalized differential expression analysis in a genetically diverse population of mice, are relevant to human HF. By linking those genes both to upstream regulators and to a signaling network predictive of cardiac hypertrophy, new insights are provided into the regulation of the severity of and resistance to cardiac hypertrophy at the individual level, and validate Hes1 as a novel regulator of cardiac hypertrophy in vitro. This approach is likely critically important for the appropriate design of upcoming experiments directed at unraveling causal genes in complex diseases.

Methods

Overview of the HMDP

The hybrid mouse diversity panel (HMDP) consists of a population of over 100 inbred mouse strains selected for usage in systematic genetic analyses of complex traits. Strain were selected to increase resolution of genetic mapping with a renewable resource that is available to all investigators worldwide as well as to create a shared data repository that would allow the integration of data across multiple scales, including genomic, transcriptomic, metabolomic, proteomic, and clinical phenotypes. The core of the panel for association mapping consists of 29 classic parental inbred strains which are a subset of a group of mice commonly called the mouse diversity panel. HMDP strains were chosen by eliminating closely related strains and removing wild-derived strains. The decision to remove wild-derived stains reflects a tradeoff between statistical power and genetic diversity. While leaving out wild-derived strains sacrifices genetic diversity to some degree, the HMDP increased the statistical power (assuming the same number of animals) to identify genetic variants polymorphic among the classical inbred strains which affect traits. These variants yield a tremendous amount of phenotypic diversity among the classical inbred strains.

ISO Treatment

30 mg per kg body weight per day of Isoproterenol (ISO) was administered for 21 days in 8-10 week old female mice using ALZET osmotic mini-pumps, which were surgically implanted intraperitoneally. All animal experiments were conducted following guidelines established and approved by the University of California, Los Angeles Institutional Animal Care and Use Committee (IACUC) and housed in an IACUC-approved vivarium with daily monitoring by vivarium personnel.

Hypertrophy Measurement

As each mouse in a strain is genetically identical, several mice from the same strain were used for measuring the cardiac hypertrophic response to ISO treatment. More specifically, on average three untreated mice were used serving as control hearts and about three ISO treated mice of the same strain were used to measure the cardiac hypertrophic response. This response was studied in a total of 104 genetically different strains with the precise number of control and treated hearts for each strain. The number of untreated control hearts per strain was 2.75. The average number of ISO treated hearts per strain was 3.5. At sacrifice, hearts were excised, drained of excess blood and weighed. Each of the four chambers of the heart (left ventricle with inter-ventricular septum, right-ventricular free wall, right and left atria) was isolated and subsequently weighed. Cardiac hypertrophy for a given strain was calculated as the increase in average total heart weight after ISO treatment compared to control mice.

Heart Biopsy for Microarray Analysis

As for the hypertrophy measurement, the fact that each mouse in a strain is genetically identical could be exploited to extract heart tissue for microarray analysis in both untreated and ISO treated mice from the same strain. The left ventricle of each heart was cut into quarters with each piece weighing on average about 25 mg+/−a few mg depending on the amount of hypertrophy and two pieces were used for microarray data analysis. Due to the large number of strains analyzed and the cost of microarray data analysis, one untreated control heart and one ISO treated heart was used per strain for about 90% of the strains. However, since mice of a given strain are renewable, the HMPD offers the possibility to use triplets, quadruplets, and higher multiples of isogenic subjects for experimentation. This feature was used to measure gene expression in replicates (e.g., two hearts in control or two hearts after ISO treatment) to test for replicability in about ten percent of the strains.

Microarray Data Analysis

Following homogenization of left ventricular tissue samples in QIAzol, RNA was extracted using the Qiagen miRNAeasy extraction kit, and verified as having a RIN>7 by Agilent Bioanalyzer. Two RNA samples were pooled for each strain and experimental condition and arrayed on Illumina Mouse Reference 8 version 2.0 chips. Analysis was conducted using the Neqc algorithm included in the limma R package and batch effects addressed using COMbat. In designing the study, the treated and control conditions were distributed evenly across the three batches as well as endeavoring to include a diverse set of genetic backgrounds in each batch. Thus, the data likely does not suffer from potential batch artifacts. Microarray data may be accessed at the Gene Expression Omnibus using accession ID: GSE48760. All phenotypic and expression data may also be accessed at https://systems.genetics.ucla.edu/data/hmdp_hypertrophy_heart_failure.

Overview of the Gene Correlation Method

Traditional analyses of differential gene expression for complex diseases rely on gene expression data for two populations: a control population and a diseased (or drug treated) population. For example, in the case of HF, the control population consists of N donors with healthy hearts intended to be used for transplantation, which are biopsied for gene expression analysis when left unused, and the diseased population consists of M late stage heart failure patients whose hearts are explanted and then biopsied for gene expression analysis. Importantly, the subjects in the control and diseased population are all genetically different. Hence, if the subjects were labeled by si, where the index i refers to subject i with its own genetic background distinct from all other subjects, the N subjects in the control population are (S₁, S₂, . . . , S_{N}) (control subjects) and the M subjects in the diseased population are (S_{N+1}, S_{N+2} . . . S_{N+M}) (diseased subjects).

The data sets used for the differential gene expression analysis consists then of the expression level (log 2 mRNA number) of a large number of K genes for each subject. K is typically in the range of several thousands, and thus much larger than the number of control or diseased subjects (N or M, respectively) that are at most a few hundreds in the most extensive studies to date, and only a few subjects in each population in earlier studies. Let us label the expression levels by Eᵢ(1) where the subscript i refers to subject i and the index j=1, K refers to gene j. To find out if a given gene j among the K genes is differentially expressed, it suffices to use a standard statistical test analogous to a student T-test to decide if the gene expression data for the control group (E₁(j), E₂(j), . . . , E_{N}(j)) (expression data for gene j in control population) and for the diseased group (E_{N+1}(j), E_{N+2}), . . . , E_{N+M}( )) (expression data for gene j in genetically distinct diseased population) originate from the same or different distributions. This test is carried out for all K genes and differentially expressed genes are then ranked in order of statistical significance (e.g., with increasing p-value less than some threshold of statistical significance). This approach is well-established and can be performed using existing bioinformatics tools such as SAM (Statistical Analysis of Microarrays).

Because mice from the same strains are isogenic and renewable, the HMDP offers the possibility to analyze differential gene expression in a different and unique setting where subjects in the control and diseased populations have the same genetic background. The control population consists of one mouse per strain (for N strains) before treatment with a beta-adrenergic agonist isoproterenol (ISO) inducing cardiac hypertrophy and heart failure. Since all strains are genetically distinct the subjects in the control population are genetically distinct and can be labelled as (S₁, S₂, . . . , S_{N}) (genetically identical control and diseased populations in the HMDP). Hearts from those subjects before ISO treatment are biopsied and used for microarray analysis. Biopsy requires sacrificing the animals that cannot be ISO treated. However, another mouse from the same strain can be ISO treated and similarly for all N strains. Therefore, the diseased/treated population is genetically identical to the control population and has the same degree of genetic diversity, leaving aside for now epigenetic mechanisms affecting gene expression and trait that are discussed further below.

From the gene expression data alone, the standard SAM type of differential gene expression analysis can be performed that consists of deciding if the gene expression data before (E₁(j), E₂(j), . . . , E_{N}(j)) (expression data for gene j in control strains) and after (E′₁(j), E′₂(j), . . . , E′_{N}(i)) (expression data for gene j in genetically identical treated strains) treatment originate from the same or different distributions, where Eᵢ(j) and E′ᵢ(j) are the expression levels of gene j for the isogenic subjects si before (in control) and after ISO treatment, respectively. Those HMDP genes may be referred to as SAM genes.

One can also perform an entirely novel type of differential gene expression analysis owing to the fact that, in addition to control and treated subjects belonging to the same strain having the same genetic background, the change of heart mass in response to ISO, i.e., the ratio m′ᵢ/mᵢ of total heart mass before (mᵢ) and after ISO treatment (m′ᵢ) for strain i, can be measured for all strains (i=1, 2, . . . , N) to assess the degree of hypertrophy among different strains. This ratio is calculated by measuring total heart mass for several mice from the same strain before and after ISO treatment and averaging measured values before and after ISO treatment prior to taking their ratio. Importantly, values of m′ᵢ/mᵢ range continuously from about 1 (no change of heart mass) to 2 (two-fold change of heart mass) among strains. Differential gene expression can then be examined by asking whether a given gene j contributes to the severity of cardiac hypertrophy. This can be readily done by calculating the coefficient of correlation c₁ (e.g., Pearson or Spearman) between the strain-specific fold change of expression of gene j in response to ISO treatment among different strains

$${F}_{j} = ( \frac{{E}_{1}^{′} ( j )}{{E}_{1} ( j )} , \frac{{E}_{2}^{′} ( j )}{{E}_{2} ( j )} , … , \frac{{E}_{N}^{′} ( j )}{{E}_{N} ( j )} )$$

and the strain-specific change of heart mass among different strains

$${F}_{m} = ( \frac{{m}_{1}^{′}}{{m}_{1}} , \frac{{m}_{2}^{′}}{{m}_{2}} , … , \frac{{m}_{N}^{′}}{{m}_{N}} ) .$$

Clearly, this correlation coefficient cannot be calculated in the setting of traditional clinical studies since the fold change of gene expression or heart mass of subjects with different genetic background is meaningless. Calculating this correlation would require using a population of identical twins in which one twin for each pair of twins is a heart donor and the other twin is a late stage HF patient, and donor and explanted hearts could be biopsied.

The HMDP provides the experimental tool to carry out this identical twins experiment to measure expression data and trait (heart mass) for the same genetic background under different conditions (before and after ISO treatment). The correlation coefficients cⱼ can be positive or negative and the magnitude of cⱼ can be used to identify genes and classify them in order of statistical significance assessed by comparing cⱼ values computed with actual data to those computed with a randomized data set (e.g., a set obtained by permuting the strain labels). Genes identified by this method can be referred to as fold-change (FC) genes to reflect the fact that they are obtained by correlating the individual fold-change of gene expression for all strains (Fⱼ) with the individual fold-change of heart mass for all strains (Fₘ).

In this conceptual “identical twin” experiment, only two mice per strain are used for microarray data analysis in 90% of the strains (one control mouse and one treated mouse). This experimental limitation stems from the large number of hearts (over 200) that need to be biopsied and analyzed for gene expression. However, since mice of a given strain are renewable, the HMPD offers the possibility to use triplets, quadruplets, and higher multiples of isogenic subjects for experimentation. This feature was used to measure gene expression in replicates (e.g., two hearts in control or two hearts after ISO treatment) to test for replicability in about ten percent of the strains. The results of this replicability analysis show that genetics play a dominant role in controlling gene expression and that using two mice per strain (on in the control group and one in the treated group) is sufficient to identify FC genes. This conclusion is further supported by the fact that, remarkably, the FC genes turn out to be for the most part completely different than the traditional SAM genes, and causally related to hypertrophy as assessed by further analysis of pathway enrichment and direct experimental validation of the role of one FC gene.

Pre-Filtering of the Data

In order to reduce false positive predictions and computational time, the 25,697 genes expression data can be filtered. Instead of setting an arbitrary cutoff based on the level of expression as is commonly done, a network approach can be used that is consistent with the correlation-based methods used in this study. The idea is that the different genotypic backgrounds across strains lead to global gene expression modulation, thus creating correlation between expressed genes. Genes not associated with the core of varying genes should be the ones that carry too much experimental noise due to low expression or systematic biases.

First, the absolute Pearson correlation was computed for gene expression fold-change between all pairs of genes. This creates a complete weighted network containing all genes. Genes for which expression is noisy because of low expression or experimental artifacts should have a low association to the other genes. Therefore, the size of the Largest Connected Component (LCC) of the network was evaluated when hard-thresholding with several correlation cutoffs. A fast decrease was observed of the LCC size at low thresholds of 0.35-0.45, followed by milder steady decrease. A cutoff was used of 0.5 corresponding to that stabilization plateau and kept the 11,279 genes in the LCC. The effect of this filter is made clear by looking at a selection of functional genes linked to the electromechanical coupling in heart cells. The rejected genes (gray bars) have either low expression (e.g., Calm4, Kcnd3) or display systematic saturation effects inherent to the microarray assay, which results in noisy correlations (e.g., Tnnc1, Atp2a2). These 11,279 genes can be used as input to the different methods.

Computation of Randomized Correlations

To compute the expected correlations of FIG. 2A, the heart mass fold-changes can be shuffled among strains. The correlations are then computed between all genes FCs and this randomized phenotype. The step can be repeated, for example, 1,000 times. The final histogram is the average over the 1,000 randomizations.

Computation of Population-Wide DEGs

The population-wide DEGs are computed by using Significance Analysis of Microarray or SAM between the post-ISO and the pre-ISO expression data. Using a False Discovery Rate of 1e-3, 2,538 significant DEGs were found.

Conversion from Mouse Symbols to Human Entrez IDs

In order to compute pathway and disease genes enrichment, a table converting mouse gene symbols to human entrez IDs was computed. The UCSC genome browser mm9.kgXref, mm9.hgBlastTab and hg19.kgXref conversion tables, available on the mySQL host genome-mysql.cse.ucsc.edu, was used. The kgXref tables were used for conversion between symbols and entrez IDs while the Blast table was used to get the human orthologs of mouse genes.

HuGE Database

Disease genes were taken from the HuGE database of published GWAS genes, with a total of 2,711 diseases. HF related diseases were filtered out using keywords ‘heart’, ‘cardi’, ‘hypert’, ‘aort’, ‘fibro’.

Pathways

Pathways were taken from MSigDB v3.1 and Wikipathways, with a total of 8,690 sets of genes. A group of 106 genes corresponding to a previously published Cardiac Hypertrophic Signaling Network (CHSN) was added under the name “SAUCERMAN_cardiac_hypertrophy_pathway”.

TF Enrichment

The cytoscape plugin iRegulon was used to predict putative upstream TF regulating the studied sets of genes. Default parameters were used: 9,713 PWMs scanning 20 kb centered around TSS.

Computation of Statistics

All statistics (correlations, t-test, Wilcoxon test, hypergeometric test) were computed using R. Hierarchical clustering was performed using default parameters of the R hclust function. Z scores correspond to the number of standard deviations a given observation is away from the mean of the null (random) distribution and are computed as follows:

$$Z = \frac{x - 〈 X 〉}{〈 {( X - 〈 X 〉 )}^{2} 〉}$$

where x is the observed value, X is a set of random predictions, and <.> denotes the average.

Cell Culture and Treatments

Right ventricular myocytes were isolated and cultured, as reported using 2-4 day old rats. Myocytes and fibroblasts were separated with Percoll density gradient. For knockdown experiments cells were transfected with Hes1 siRNA using lipofectamine RNAimax (life technologies).

RNA Isolation and qPCR

RNA isolation from cells was performed using Qiazol lysis reagent. cDNA synthesis was performed using the High Capacity Reverse Transcription cDNA Kit (Life Technologies). qPCR was performed using the LightCycler 480 (Roche).

Quantification of Cardiomyocyte Cell Cross-Sectional Area

Quantification of cardiomyocyte cell cross-sectional area was done following transfection with either control or Hes1 siRNA and a 48-hour treatment with control or isoproterenol or phenylepherine containing media. Images were taken on a Nikon Eclipse TE2000-U microscope. Images were analyzed using the Nikon Imagine System (NIS). 150 cells were used to compute the SEM.

FIG. 5 is a flow diagram illustrating a method 500 of identifying relevant genes correlated to phenotypic responses to treatment of complex diseases, according to an example embodiment. The example method 500 includes acquiring 505 data, for a plurality of individuals, representing biomolecular expressions 510 and phenotypic responses 515 to a treatment. The data for each individual includes a biomolecular expression and phenotypic response pair. The method 500 further includes calculating 520, across the plurality of individuals, correlation coefficients between the biomolecular expressions and the phenotypic responses to obtain observed correlation coefficients. The method 500 further includes randomizing 525 pairings between the biomolecular expressions and the phenotypic responses and recalculating the coefficients to obtain randomized correlation coefficients. The observed correlation coefficients are compared 530 with the randomized correlation coefficients to create enrichment data, and, based on the enrichment data and the biomolecular expressions, a plurality of relevant genes correlated to the phenotypic responses are identified 535.

Example Digital Processing Environment

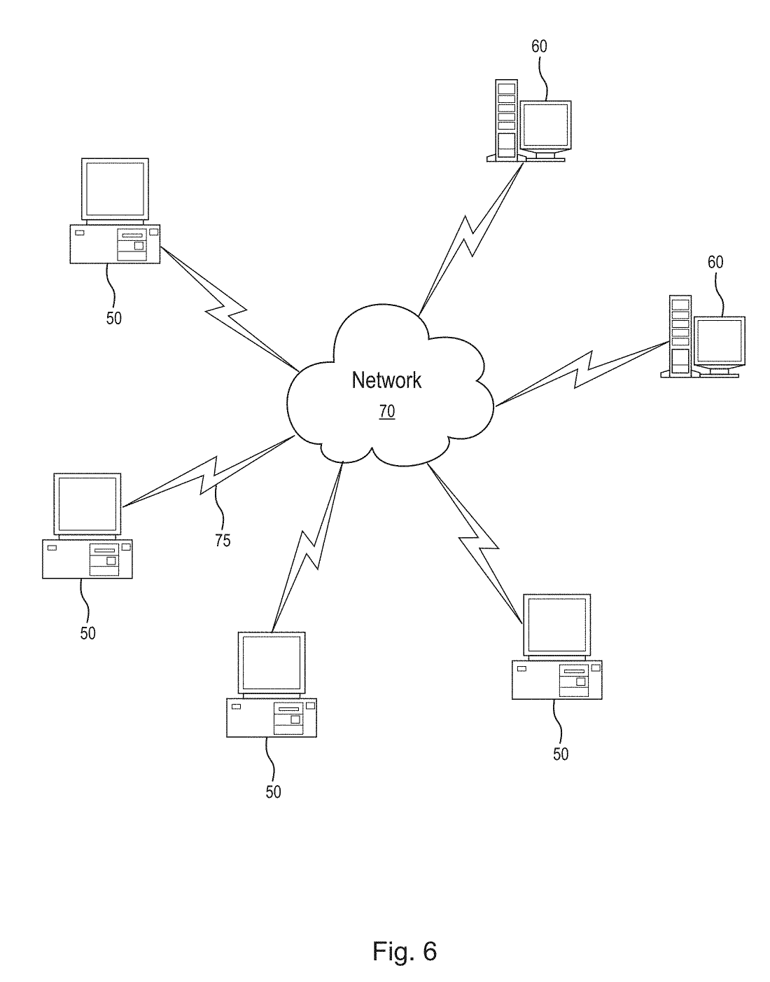

FIG. 6 illustrates a computer network or similar digital processing environment in which the present embodiments may be implemented. Client computer(s)/devices 50 and server computer(s) 60 provide processing, storage, and input/output devices executing application programs and the like. Client computer(s)/devices 50 can also be linked through communications network 70 to other computing devices, including other client devices/processes 50 and server computer(s) 60. Communications network 70 can be part of a remote access network, a global network (e.g., the Internet), cloud computing servers or service, a worldwide collection of computers, Local area or Wide area networks, and gateways that currently use respective protocols (TCP/IP, Bluetooth, etc.) to communicate with one another. Other electronic device/computer network architectures are suitable.

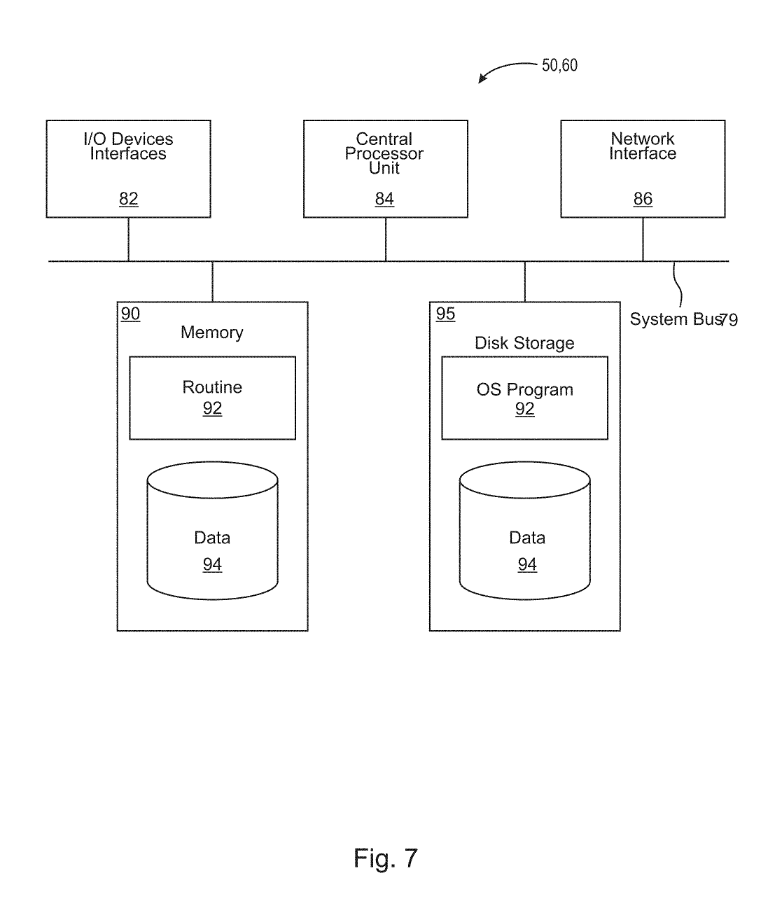

FIG. 7 is a diagram of the internal structure of a computer (e.g., client processor/device 50 or server computers 60) in the computer system of FIG. 6. Each computer 50, 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, and network ports) that enables the transfer of information between the elements. Attached to system bus 79 is I/O device interface 82 for connecting various input and output devices (e.g., keyboard, mouse, displays, printers, and speakers) to the computer 50, 60. Network interface 86 allows the computer to connect to various other devices attached to a network (e.g., network 70 of FIG. 6). Memory 90 provides volatile storage for computer software instructions 92 and data 94 used to implement many embodiments (e.g., the example method 500 of FIG. 5). Disk storage 95 provides non-volatile storage for computer software instructions 92 and data 94 used to implement many embodiments. Central processor unit 84 is also attached to system bus 79 and provides for the execution of computer instructions.

In the context of FIG. 6, the computer 50, 60 can be used to acquire (via, for example, interface 82) data representing biomolecular expressions and phenotypic responses to a treatment. Components of the system include memory 90, 95 and a processor 84. The memory 90, 95 can store the data representing biomolecular expressions and phenotypic responses. The processor 84 can be configured to calculate correlation coefficients between the biomolecular expressions and the phenotypic responses to obtain observed correlation coefficients, randomize pairings between the biomolecular expressions and the phenotypic responses and recalculate the coefficients to obtain randomized correlation coefficients, compare the observed correlation coefficients with the randomized correlation coefficients to create enrichment data, and identify, based on the enrichment data and the biomolecular expressions, a plurality of relevant genes correlated to the phenotypic responses.

In one embodiment, the processor routines 92 and data 94 are a computer program product (generally referenced 92), including a computer readable medium (e.g., a removable storage medium such as one or more DVD-ROM's, CD-ROM's, diskettes, and tapes) that provides at least a portion of the software instructions for the system. Computer program product 92 can be installed by any suitable software installation procedure, as is well known in the art. In another embodiment, at least a portion of the software instructions may also be downloaded over a cable, communication and/or wireless connection. In other embodiments, the program product 92 may be implemented as Software as a Service (SaaS), or other installation or communication supporting end-users.

While example embodiments have been particularly shown and described, it will be understood by those skilled in the art that various changes in form and details may be made therein without departing from the scope of the embodiments encompassed by the appended claims. For example, while a particular example has been disclosed that uses mice from the Hybrid Mouse Diversity Panel, other subjects may be used, such as stem cells for an individual that have been replicated. For example, stem cells from an individual and be replicated and reprogrammed to be of a certain type of cell (e.g., cardiac cell). These cells can be the subject of the methods and systems disclosed herein.

What is claimed is:

1. A method of identifying relevant genes correlated to phenotypic responses to treatment of complex diseases, the method comprising:
acquiring data, for a plurality of individuals, representing biomolecular expressions and phenotypic responses to a treatment, the data for each individual including a biomolecular expression and phenotypic response pair;
calculating, across the plurality of individuals, correlation coefficients between the biomolecular expressions and the phenotypic responses to obtain observed correlation coefficients;
randomizing pairings between the biomolecular expressions and the phenotypic responses and recalculating the coefficients to obtain randomized correlation coefficients;
comparing the observed correlation coefficients with the randomized correlation coefficients to create enrichment data; and
identifying, based on the enrichment data and the biomolecular expressions, a plurality of relevant genes correlated to the phenotypic responses.

2. The method of claim 1 wherein:
the biomolecular expression for an individual includes biomolecular expression data before treatment of the individual;
the phenotypic response for an individual is computed from measurements of the phenotype before and after treatment of the individual; and
calculating the correlation coefficients includes calculating correlation coefficients between the biomolecular expression data before treatment of the individuals and the phenotypic responses.

3. The method of claim 1 wherein:
the biomolecular expression for an individual includes a fold change of biomolecular expression data before and after treatment of the individual, the fold change being a ratio of the biomolecular expression after and before treatment of the individual;
the phenotypic response for an individual is computed from measurements of the phenotype before and after treatment of the individual; and
calculating the correlation coefficients includes calculating correlation coefficients between the fold changes of biomolecular expression data and the phenotypic responses.

4. The method of claim 1 wherein the phenotypic responses are either binary or of a continuous spectrum of phenotypic responses.

5. The method of claim 1 wherein acquiring data includes acquiring biomolecular expressions from the individuals and evaluating phenotypic responses of the individuals before and after treatment.

6. The method of claim 1 further comprising filtering the biomolecular expressions to exclude biomolecular expressions that are of lower confidence.

7. The method of claim 1 wherein comparing the observed correlation coefficients with the randomized correlation coefficients to create enrichment data includes dividing a proportion of the observed correlations by a proportion of the randomized correlations.

8. The method of claim 1 wherein the enrichment data includes an enrichment curve as a function of the observed correlation coefficients ordered by decreasing values.

9. The method of claim 8 wherein identifying the plurality of genes relevant to the phenotypic responses includes identifying the plurality of genes based on observed correlation coefficients from the start of the enrichment curve to the peak of the enrichment curve.

10. A system for identifying relevant genes correlated to phenotypic responses to treatment of complex diseases, the system comprising:
an interface configured to acquire data, for a plurality of individuals, representing biomolecular expressions and phenotypic responses to a treatment, the data for each individual including a biomolecular expression and phenotypic response pair;
memory storing the data; and
a processor in communication with the interface and memory, and configured to:
calculate, across the plurality of individuals, correlation coefficients between the biomolecular expressions and the phenotypic responses to obtain observed correlation coefficients;
randomize pairings between the biomolecular expressions and the phenotypic responses and recalculate the coefficients to obtain randomized correlation coefficients;
compare the observed correlation coefficients with the randomized correlation coefficients to create enrichment data; and
identify, based on the enrichment data and the biomolecular expressions, a plurality of relevant genes correlated to the phenotypic responses.

11. The system of claim 10 wherein:
the biomolecular expression for an individual includes biomolecular expression data before treatment of the individual;
the phenotypic response for an individual is computed from measurements of the phenotype before and after treatment of the individual; and
the processor is configured to calculate the correlation coefficients based on the biomolecular expression data before treatment of the individuals and the phenotypic responses.

12. The system of claim 10 wherein:
the biomolecular expression for an individual includes a fold change of biomolecular expression data before and after treatment of the individual, the fold change being a ratio of the biomolecular expression after and before treatment of the individual;
the phenotypic response for an individual is computed from measurements of the phenotype before and after treatment of the individual; and
the processor is configured to calculate the correlation coefficients based on the fold changes of biomolecular expression data and the phenotypic responses.

13. The system of claim 10 wherein the phenotypic responses are either binary or of a continuous spectrum of phenotypic responses.

14. The system of claim 10 wherein the interface enables acquisition of biomolecular expressions from the individuals and evaluation of phenotypic responses of the individuals before and after treatment.

15. The system of claim 10 wherein the processor is configured to filter the biomolecular expressions to exclude biomolecular expressions that are of lower confidence.

16. The system of claim 9 wherein the processor is configured to create the enrichment data by dividing a proportion of the observed correlations by a proportion of the randomized correlations.

17. The system of claim 10 wherein the enrichment data includes an enrichment curve as a function of the observed correlation coefficients ordered by decreasing values.

18. The system of claim 17 wherein the processor is configured to identify the plurality of genes relevant to the phenotypic responses based on observed correlation coefficients from the start of the enrichment curve to the peak of the enrichment curve.

19. A machine-readable storage medium having stored thereon a computer program for identifying relevant genes correlated to phenotypic responses to treatment of complex diseases, the computer program comprising a routine of set instructions for causing the machine to:
acquire data, for a plurality of individuals, representing biomolecular expressions and phenotypic responses to a treatment, the data for each individual including a biomolecular expression and phenotypic response pair;
calculate, across the plurality of individuals, correlation coefficients between the biomolecular expressions and the phenotypic responses to obtain observed correlation coefficients;
randomize pairings between the biomolecular expressions and the phenotypic responses and recalculate the coefficients to obtain randomized correlation coefficients;
compare the observed correlation coefficients with the randomized correlation coefficients to create enrichment data; and
identify, based on the enrichment data and the biomolecular expressions, a plurality of relevant genes correlated to the phenotypic responses.

20. The machine-readable storage medium of claim 19 wherein:
the biomolecular expression for an individual includes biomolecular expression data before treatment of the individual;
the phenotypic response for an individual is computed from measurements of the phenotype before and after treatment of the individual; and
the instructions cause the processor to calculate the correlation coefficients based on the biomolecular expression data before treatment of the individuals and the phenotypic responses.

21. The machine-readable storage medium of claim 19 wherein:
the biomolecular expression for an individual includes a fold change of biomolecular expression data before and after treatment of the individual, the fold change being a ratio of the biomolecular expression after and before treatment of the individual;
the phenotypic response for an individual is computed from measurements of the phenotype before and after treatment of the individual; and
the instructions cause the processor to calculate the correlation coefficients based on the fold changes of biomolecular expression data and the phenotypic responses.

22. The machine-readable storage medium of claim 19 wherein the phenotypic responses are either binary or of a continuous spectrum of phenotypic responses.

23. The machine-readable storage medium of claim 19 wherein the instructions cause the processor to enable acquisition of biomolecular expressions from the individuals and evaluation of phenotypic responses of the individuals before and after treatment.

24. The machine-readable storage medium of claim 19 wherein the instructions cause the processor to filter the biomolecular expressions to exclude biomolecular expressions that are of lower confidence.

25. The machine-readable storage medium of claim 19 wherein the instructions cause the processor to create the enrichment data by dividing a proportion of the observed correlations by a proportion of the randomized correlations.

26. The machine-readable storage medium of claim 19 wherein the enrichment data includes an enrichment curve as a function of the observed correlation coefficients ordered by decreasing values.

27. The machine-readable storage medium of claim 26 wherein the instructions cause the processor to identify the plurality of genes relevant to the phenotypic responses based on observed correlation coefficients from the start of the enrichment curve to the peak of the enrichment curve.
